SEQUENCE LISTING (12 sequences)

SEQ ID NO: 1 - DNA, 24 bp, Artificial
  synthetic
tgatggtgtatgtgattttgtgtt

SEQ ID NO: 2 - DNA, 18 bp, Artificial
  Synthetic
accccacccctcacaaac

SEQ ID NO: 3 - DNA, 16 bp, Artificial
  synthetic
acctacgaacgaacac

SEQ ID NO: 4 - DNA, 17 bp, Artificial
  synthetic
tacctacaaacaaacac

SEQ ID NO: 5 - DNA, 21 bp, Artificial
  synthetic
aggggttaggttgaggttgtt

SEQ ID NO: 6 - DNA, 21 bp, Artificial
  synthetic
tctacctcttcccaaattcca

SEQ ID NO: 7 - DNA, 15 bp, Artificial
  synthetic
ttggatggatcgcgg

SEQ ID NO: 8 - DNA, 19 bp, Artificial
  synthetic
tattttggatggattgtgg

SEQ ID NO: 9 - DNA, 19 bp, Artificial
  synthetic
ggggataggaagatggttt

SEQ ID NO: 10 - DNA, 18 bp, Artificial
  synthetic
cccccatcccatacaacc

SEQ ID NO: 11 - DNA, 18 bp, Artificial
  synthetic
tttgtttcgtagcggagt

SEQ ID NO: 12 - DNA, 20 bp, Artificial
  synthetic
gattttgttttgtagtggag

(19) United States
(12) Patent Application Publication
(10) Pub. No.: US 2010/0003689 A1
(43) Pub. Date: Jan. 7, 2010

(54) Use of Methylation Status of MINT Loci as a Marker for Rectal Cancer

(75) Inventors: Dave S. B. HOON, Santa Monica, CA (US); Michiel F.G. de Maat, Santa Monica, CA (US)

Correspondence Address: HOGAN & HARTSON L.L.P., 1999 AVENUE OF THE STARS, SUITE 1400, LOS ANGELES, CA, 90067, US

(73) Assignee: John Wayne Cancer Institute, Santa Monica, CA (US)

(21) Appl. No.: 12/488,494

(22) Filed: Jun. 19, 2009

Related U.S. Application Data
(60) Provisional application No. 61/074,091, filed on Jun. 19, 2008.

(51) Int. Cl. C12Q 1/68

(52) U.S. Cl. 435/6

(57) ABSTRACT

The invention relates to methods for predicting the outcome of rectal cancer and stratifying a rectal cancer treatment according to the level of DNA methylation at MINT 1, 3, 12, or 17. Also disclosed is a method of detecting rectal adenoma or malignancy based on the level of DNA methylation at MINT 2, 3, or 31.

### RELATED APPLICATION

This application claims priority to U.S. Provisional Application Ser. No. 61/074,091, filed on Jun. 19, 2008, the content of which is incorporated herein by reference in its entirety.

### FIELD OF THE INVENTION

The present invention relates in general to the MINT (methylated-in-tumor) loci. More specifically, the invention relates to the use of the methylation status of some specific MINT loci as a biomarker for diagnosis, prognosis, and treatment of rectal cancer.

### BACKGROUND OF THE INVENTION

Rectal cancer is the second most common cancer of the digestive system in the U.S.A.¹ Neoadjuvant therapy has improved local control of rectal cancer in patients undergoing total mesorectal excision (TME),²⁻⁴ but distant recurrence remains the major cause of disease mortality. Although tumor status of regional nodes is the most important predictor of metastasis, 20% of node-negative patients will recur at distant sites. This suggests that even early stages of tumors have potential for systemic metastasis and, therefore, molecular subclassification may be clinically relevant. Development of prognostic molecular biomarkers for rectal cancer would improve management and potential treatment stratification. Colon and rectal cancers are often assessed together in the analysis of molecular/genetic biomarkers. This is often due to the limited availability of tumor for analysis, or specimens are not procured from a specific clinical trial. We now know both cancers are different in etiology and treatment, as well as (epi)genetics.⁵

### SUMMARY OF THE INVENTION

The present invention is based, at least in part, upon the unexpected discovery that the methylation status of MINT loci such as MINT 1, 2, 3, 12, 17, or 31 can be used as a biomarker for diagnosis, prognosis, and treatment of rectal cancer.

Accordingly, in one aspect, the invention features a method of detecting rectal adenoma or malignancy. The method comprises providing a test biological sample from a subject, and determining the level of DNA methylation at MINT 2, 3, or 31 in the test sample. If the level of methylation at MINT 2, 3, or 31 in the test sample is higher than that in a normal sample, the subject is likely to be suffering from rectal adenoma or malignancy.

In another aspect, the invention features a method of predicting the outcome of rectal cancer. The method comprises providing a first sample containing rectal cancer cells from a first subject, and determining the level of DNA methylation at MINT 3 and the level of DNA methylation at MINT 1, 12, or 17 in the first sample. If the level of DNA methylation at MINT 3 in the first sample is higher than that in a second sample containing rectal cancer cells from a second subject, and the level of DNA methylation at MINT 1, 12, or 17 in the first sample is lower than that in the second sample, the first subject is likely to have an increased risk for distant recurrence, a shorter cancer-specific survival, and a shorter overall survival compared to the second subject. The first or second subject may be either node-negative or node-positive.

In some embodiments, the level of DNA methylation at MINT 3 and the level of DNA methylation at MINT 17 in the first sample are determined. If the level of DNA methylation at MINT 3 in the first sample is higher than that in the second sample, and the level of DNA methylation at MINT 17 in the first sample is lower than that in the second sample, the first subject is likely to have an increased risk for distant recurrence, a shorter cancer-specific survival, and a shorter overall survival compared to the second subject.

Another method of predicting the outcome of rectal cancer comprises providing a first sample containing rectal cancer cells from a first subject, and determining the level of DNA methylation at MINT 3 and the level of DNA methylation at MINT 1, 12, or 17 in the first sample. If the level of DNA methylation at MINT 3 in the first sample is lower than that in a second sample containing rectal cancer cells from a second subject, and the level of DNA methylation at MINT 1, 12, or 17 in the first sample is higher than that in the second sample, the first subject is likely to have an increased risk for local recurrence compared to the second subject.

In some embodiments, the level of DNA methylation at MINT 3 and the level of DNA methylation at MINT 17 in the first sample are determined. If the level of DNA methylation at MINT 3 in the first sample is lower than that in the second sample, and the level of DNA methylation at MINT 17 in the first sample is higher than that in the second sample, the first subject is likely to have an increased risk for local recurrence compared to the second subject.

In some embodiments, the first subject is likely to have an increased risk for local recurrence compared to a third subject that suffers from rectal cancer and receives a radiation therapy prior to a mesorectal excision (ME) for primary rectal cancer.

In some embodiments, if the second subject is node-positive, this subject is likely to have an increased risk for local recurrence compared to a third subject that suffers from rectal cancer but is node-negative.

In the methods described above, the first subject may not receive a radiation therapy prior to an ME for primary rectal cancer, the rectal cancer cells may be primary rectal cancer cells, or the first or second subject may be suffering from an AJCC Stage I, II, or III rectal cancer.

The invention also provides a method of stratifying a rectal cancer treatment. The method comprises providing a biological sample containing primary rectal cancer cells from a subject prior to an ME for primary rectal cancer and a radiation therapy, determining the level of DNA methylation at MINT 3 and the level of DNA methylation at MINT 17 in the sample, and stratifying a rectal cancer treatment according to the level of DNA methylation at MINT 3 and the level of DNA methylation at MINT 17 in the sample.

In some embodiments, if the subject is in a cluster of subjects with rectal cancer that have increased level of DNA methylation at MINT 3 and decreased level of DNA methylation at MINT 17 compared to other clusters of subjects with rectal cancer, no radiation therapy is to be given to the subject prior to the ME.

In some embodiments, if the subject is in a cluster of subjects with rectal cancer that have decreased level of DNA methylation at MINT 3 and increased level of DNA methylation at MINT 17 compared to other clusters of subjects with rectal cancer, a radiation therapy is to be given to the subject prior to the ME.

The clusters of subjects with rectal cancer may be determined, e.g., using unsupervised random forest (RF) clustering and an expectation-maximization mixture of Gaussians (EM-MoG) algorithm.

A sample in the methods described above may be a rectal tissue sample or a rectal cancer sample. The level of DNA methylation at MINT 1, 2, 3, 12, 17, or 31 may be determined by absolute quantitative analysis of methylated alleles (AQAMA).

Unless otherwise defined, all technical and scientific terms used herein have the same meaning as commonly understood by one of ordinary skill in the art to which this invention pertains. In case of conflict, the present document, including definitions, will control. The materials, methods, and examples disclosed herein are illustrative only and not intended to be limiting. Other features, objects, and advantages of the invention will be apparent from the description and the accompanying drawings, and from the claims.

### BRIEF DESCRIPTION OF THE FIGURES

FIG. 1. Scatterplots of measured methylation indices in normal rectal epithelium, rectal adenoma tissue and rectal cancer tissue for the 7 MINT loci studied. (ns=non significant).

FIG. 2. MINT locus methylation subclass identification. A, MDS plot displaying (2-D) the level of dissimilarity between all patients (MDS plot axes are dimensionless representing arbitrary units). B, 3-D plot representing EM-MoG analysis of the 2-D MDS plot coordinates showing Gaussian distribution (bell-shaped) of the two identified clusters. C, MDS plot showing final cluster allocation of the patients. D, Boxplots comparing difference in methylation levels between cluster 1 and 2 for all MINT loci.

FIG. 3. Kaplan Meier plots grouping analyzed node-negative TME trial patients into cluster 1 and cluster 2 and comparing postoperative distant recurrence free survival probability (in A). In B and C cancer-specific and overall survival, respectively are plotted.

FIG. 4. Random forest analyses using only MINT3 and 17 quantitative methylation data as input. In A the MDS-plot shows four clusters. In B, boxplots show the difference in methylation levels between the four clusters. The Kaplan Meier plot in C compares distant recurrence probability between the high-risk cluster 3 and the combined clusters 1, 2, and 4.

FIG. 5. Random forest clustering using all MINT loci. (A) Multidimensional scaling (MDS) plot displaying the level of dissimilarity between all patients based on methylation levels of seven MINT loci. The dots depicted in the MDS plot represent individual patients. The Y- and X-axis represent arbitrary values. (B) Three-dimensional plot depicting expectation maximization mixture of Gaussians (EM-MoG) analysis of the MDS plot coordinates.

FIG. 6. Methylated-in-tumor (MINT) locus methylation profile identification. (A) MDS plot showing final allocation of all patients into two clusters based on MINT locus methylation profiles. (B) Box plots showing differences in methylation index (MI) of all MINT loci between the two allocated clusters.

FIG. 7. Random forest clustering using only MINT3 and MINT17. (A) MDS plot displaying allocation of patients to four separate clusters based on quantitative methylation data of MINT3 and MINT17. (B) Box plots showing differences in methylation index (MI) of MINT3 and MINT17 between the four allocated clusters.

FIG. 8. Kaplan-Meier curves displaying survival probabilities for the four allocated clusters. (A) Kaplan-Meier curve showing local recurrence free survival probabilities for the four separate clusters. (B) Kaplan-Meier curve showing local recurrence free survival probabilities for cluster 3 compared to the combined clusters 1, 2 and 4.

FIG. 9. Local recurrence probability.

### DETAILED DESCRIPTION OF THE INVENTION

The present invention relates to diagnosis, prognosis, and treatment of rectal cancer using DNA methylation at MINT 1, 2, 3, 12, 17, or 31 as a biomarker.

MINT 1, 2, 3, 12, 17, and 31 are known in the art. For example, the GenBank Accession Numbers for the human MINT 1, 2, 3, 12, 17, and 31 are:

| | |
|---|---|
| MINT 1 | AF135501 |
| MINT 2 | AF135502 |
| MINT 3 | AF135503 |
| MINT 12 | AF135512 |
| MINT 17 | AF135517 |
| MINT 31 | AF135531 |

One object of the invention is to provide a method for diagnosing rectal adenoma or malignancy. A test biological sample from a subject is provided. As used herein, a “subject” refers to a human or animal, including all mammals such as primates (particularly higher primates), sheep, dog, rodents (e.g., mouse or rat), guinea pig, goat, pig, cat, rabbit, and cow. In a preferred embodiment, the subject is a human. In another embodiment, the subject is an experimental animal or animal suitable as a disease model.

The test sample may be obtained from tissues where rectal cancer may originate or metastasize. Such tissues are known in the art. For example, it is well known that rectal cancer may originate from rectal epithelium lining of the large bowel, and metastasize to liver, and other tissues.

The test sample may also be obtained from body fluids where rectal cancer cells may be present. Such body fluids are also known in the art, including, without limitation, blood, serum, plasma, bone marrow, cerebral spinal fluid, peritoneal/pleural fluid, lymph fluid, ascite, serous fluid, sputum, lacrimal fluid, stool, and urine.

A test sample may be prepared using any of the methods known in the art. Methods for extracting cellular DNA are well known in the art. Typically, cells are lysed with detergents. After cell lysis, proteins are removed from DNA using various proteases. DNA is then extracted with phenol, precipitated in alcohol, and dissolved in an aqueous solution.

The level of DNA methylation at MINT 2, 3, or 31 in the test sample is determined. DNA methylation can be detected and quantified by any method commonly used in the art, for example, methylation-specific PCR (MSP), bisulfite sequencing, pyrosequencing, and AQAMA.

MSP is a technique whereby DNA is amplified by PCR dependent upon the methylation state of the DNA. Determination of the methylation state of a nucleic acid includes amplifying the nucleic acid by means of oligonucleotide primers that distinguish between methylated and unmethylated nucleic acids. MSP can rapidly assess the methylation status of virtually any group of CpG sites within a CpG island, independent of the use of methylation-sensitive restriction enzymes. This assay entails initial modification of DNA by sodium bisulfite, converting all unmethylated, but not methylated, cytosines to uracils, and subsequent amplification with primers specific for methylated versus unmethylated DNA. MSP requires only small quantities of DNA, is sensitive to 0.1% methylated alleles of a given CpG island locus. MSP eliminates the false positive results inherent to previous PCR-based approaches which relied on differential restriction enzyme cleavage to distinguish methylated from unmethylated DNA. This method is very simple and can be used on small amounts of samples. MSP product can be detected by gel electrophoresis, CAE (capillary array electrophoresis), or real-time quantitative PCR.

Bisulfite sequencing is widely used to detect 5-MeC (5-methylcytosine) in DNA, and provides a reliable way of detecting any methylated cytosine at single-molecule resolution in any sequence context. The process of bisulfite treatment exploits the different sensitivity of cytosine and 5-MeC to deamination by bisulfite under acidic conditions, in which cytosine undergoes conversion to uracil while 5-MeC remains unreactive.

Exemplary AQAMA procedures are described in detail below.

The level of DNA methylation may be represented by a methylation index as a ration of the methylated DNA copy number to the sum of the methylated DNA copy number and the unmethylated DNA copy number, a ratio of the methylated DNA copy number to the unmethylated DNA copy number, or the like.

If the level of methylation at MINT 2, 3, or 31 in the test sample is higher than that in a normal sample, the subject is likely to be suffering from rectal adenoma or malignancy. As used herein, a “normal sample” is a sample prepared from a normal subject, a normal tissue such as a normal rectal epithelial tissue, or a normal body fluid.

Another object of the invention is to provide methods for predicting the outcome of rectal cancer using techniques similar to those described above.

In one method of the invention, a first sample containing rectal cancer cells from a first subject is provided. The level of DNA methylation at MINT 3 and the level of DNA methylation at MINT 1, 12, or 17 in the first sample are determined and compared with those in a second sample containing rectal cancer cells from a second subject. If the level of DNA methylation at MINT 3 in the first sample is higher than that in the second sample, and the level of DNA methylation at MINT 1, 12, or 17 in the first sample is lower than that in the second sample, the first subject is likely to have an increased risk for distant recurrence, a shorter cancer-specific survival, and a shorter overall survival compared to the second subject.

The first or second subject may be either node-negative or node-positive. By “node-negative” is meant that a cancer has not spread to the lymph nodes, as identified by histopathology. By “node-positive” is meant that a cancer has spread to the lymph nodes, as identified by histopathology.

In some embodiments, the level of DNA methylation at MINT 3 and the level of DNA methylation at MINT 17 in the first sample are determined and compared with those in the second sample. If the level of DNA methylation at MINT 3 in the first sample is higher than that in the second sample, and the level of DNA methylation at MINT 17 in the first sample is lower than that in the second sample, the first subject is likely to have an increased risk for distant recurrence, a shorter cancer-specific survival, and a shorter overall survival compared to the second subject.

In another method of the invention, a first sample containing rectal cancer cells from a first subject is provided. The level of DNA methylation at MINT 3 and the level of DNA methylation at MINT 1, 12, or 17 in the first sample are determined and compared with those in a second sample containing rectal cancer cells from a second subject. If the level of DNA methylation at MINT 3 in the first sample is lower than that in the second sample, and the level of DNA methylation at MINT 1, 12, or 17 in the first sample is higher than that in the second sample, the first subject is likely to have an increased risk for local recurrence compared to the second subject.

In some embodiments, the level of DNA methylation at MINT 3 and the level of DNA methylation at MINT 17 in the first sample are determined and compared with those in the second sample. If the level of DNA methylation at MINT 3 in the first sample is lower than that in the second sample, and the level of DNA methylation at MINT 17 in the first sample is higher than that in the second sample, the first subject is likely to have an increased risk for local recurrence compared to the second subject.

Further, the first subject is likely to have an increased risk for local recurrence compared to a third subject that suffers from rectal cancer and receives a radiation therapy prior to an ME for primary rectal cancer.

Moreover, if the second subject is node-positive, this subject is likely to have an increased risk for local recurrence compared to a third subject that suffers from rectal cancer but is node-negative.

In some embodiments, the first subject does not receive a radiation therapy prior to an ME for primary rectal cancer. In some embodiments, the rectal cancer cells are primary rectal cancer cells. In some embodiments, the first or second subject is suffering from an AJCC Stage I, II, or III rectal cancer.

The methods described above can be used to compare the outcome of rectal cancer of different subjects if the first and second samples are obtained from different subjects. On the other hand, if the first and second samples are obtained from the same subject at different time points (e.g., the first subject is a subject before a cancer treatment; the second subject is the same subject after the treatment), the method can be used to monitor the outcome of rectal cancer of the subject and evaluate the effectiveness of the treatment.

In addition, the invention provides a method for stratifying a rectal cancer treatment according to the level of DNA methylation at MINT 3 and the level of DNA methylation at MINT 1, 12, or 17.

More specifically, a biological sample containing primary rectal cancer cells is obtained from a subject prior to an ME for primary rectal cancer and a radiation therapy. The level of DNA methylation at MINT 3 and the level of DNA methylation at MINT 1, 12, or 17 in the sample are determined. If the subject is in a cluster of subjects with rectal cancer that have increased level of DNA methylation at MINT 3 and decreased level of DNA methylation at MINT 1, 12, or 17 compared to other clusters of subjects with rectal cancer, no radiation therapy is to be given to the subject prior to the ME. Alternatively, if the subject is in a cluster of subjects with rectal cancer that have decreased level of DNA methylation at MINT 3 and increased level of DNA methylation at MINT 1, 12, or 17 compared to other clusters of subjects with rectal cancer, a radiation therapy is to be given prior to the ME.

The clusters of subjects with rectal cancer can be determined using any of the methods known in the art. For example, as described in detail below, a cluster of subjects with rectal cancer that have increased level of DNA methylation at MINT 3 and decreased level of DNA methylation at MINT 17 compared to other clusters of subjects with rectal cancer (cluster 3 in Example II) and a cluster of subjects with rectal cancer that have decreased level of DNA methylation at MINT 3 and increased level of DNA methylation at MINT 17 compared to other clusters of subjects with rectal cancer (cluster 2 in Example II) by profiling subjects with rectal cancer using unsupervised RF clustering and an EM-MoG algorithm.

The discovery of DNA methylation at MINT 1, 2, 3, 12, 17, and 31 in rectal cancer cells is useful for identifying candidate compounds for treating rectal cancer. Briefly, a rectal cancer cell is contacted with a test compound. The levels of DNA methylation at MINT 1, 2, 3, 12, 17, or 31 in the rectal cancer cell prior to and after the contacting step are compared. If the level of DNA methylation in the cell changes after the contacting step, the test compound is identified as a candidate for treating rectal cancer, e.g., in view of the association of DNA methylation at MINT 1, 2, 3, 12, 17, or 31 with rectal adenoma and malignancy, as well as the risk for poor outcome of rectal cancer described above. For instance, a compound that reduces the level of DNA methylation at MINT 2, 3, or 31 may be a candidate for treating rectal adenoma and malignancy, a compound that reduces the level of DNA methylation at MINT 3 but enhances the level of DNA methylation at MINT 1, 12, or 17 may be a candidate for treating a subject with rectal cancer that has a high risk for distant recurrence, and a compound that enhances the level of DNA methylation at MINT 3 but reduces the level of DNA methylation at MINT 1, 12, or 17 may be a candidate for treating a subject with rectal cancer that has a high risk for local recurrence.

The test compounds can be obtained using any of the numerous approaches (e.g., combinatorial library methods) known in the art. Such libraries include, without limitation, peptide libraries, peptoid libraries, spatially addressable parallel solid phase or solution phase libraries, synthetic libraries obtained by deconvolution or affinity chromatography selection, and the “one-bead one-compound” libraries. Compounds in the last three libraries can be peptides, non-peptide oligomers, or small molecules. Examples of methods for synthesizing molecular libraries can be found in the art.

The compounds so identified are within the invention. These compounds and other compounds known to modulate DNA methylation can be used for treating rectal cancer by administering an effective amount of such a compound to a subject suffering from rectal cancer.

A subject to be treated may be identified in the judgment of the subject or a health care professional, and can be subjective (e.g., opinion) or objective (e.g., measurable by a test or diagnostic method such as those described above).

A “treatment” is defined as administration of a substance to a subject with the purpose to cure, alleviate, relieve, remedy, prevent, or ameliorate a disorder, symptoms of the disorder, a disease state secondary to the disorder, or predisposition toward the disorder.

An “effective amount” is an amount of a compound that is capable of producing a medically desirable result in a treated subject. The medically desirable result may be objective (i.e., measurable by some test or marker) or subjective (i.e., subject gives an indication of or feels an effect).

For treatment of cancer, a compound is preferably delivered directly to tumor cells, e.g., to a tumor or a tumor bed following surgical excision of the tumor, in order to treat any remaining tumor cells.

The compounds of the invention may be incorporated into pharmaceutical compositions. Such compositions typically include the compounds and pharmaceutically acceptable carriers. “Pharmaceutically acceptable carriers” include solvents, dispersion media, coatings, antibacterial and antifungal agents, isotonic and absorption delaying agents, and the like, compatible with pharmaceutical administration.

A pharmaceutical composition is normally formulated to be compatible with its intended route of administration. Examples of routes of administration include parenteral (e.g., intravenous), intradermal, subcutaneous, oral (e.g., inhalation), transdermal (topical), transmucosal, and rectal administration.

The dosage required for treating a subject depends on the choice of the route of administration, the nature of the formulation, the nature of the subject's illness, the subject's size, weight, surface area, age, and sex, other drugs being administered, and the judgment of the attending physician. Suitable dosages are typically in the range of 0.01-100.0 mg/kg. Wide variations in the needed dosage are to be expected in view of the variety of compounds available and the different efficiencies of various routes of administration.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### Examples

### Example I

### Quantitative Analysis of Methylation of Genomic Loci in Early Stage Rectal Cancer Predicts Distant Recurrence

### Abstract

Introduction: There are no accurate prognostic biomarkers specific for rectal cancer. Epigenetic aberrations, in the form of DNA methylation, accumulate early during rectal tumor formation. In a preliminary study, we investigated absolute quantitative methylation changes associated with tumor progression of rectal tissue at multiple genomic methylated-in-tumor (MINT) loci sequences. We then explored in a different clinical patient group whether these epigenetic changes could be correlated with clinical outcome. Methods: Absolute quantitative assessment of methylated alleles (AQAMA) was used to assay methylation changes at MINT 1, 2, 3, 12, 17, 25, and 31 in sets of normal, adenomatous and malignant tissues from 46 patients with rectal cancer. Methylation levels of these biomarkers were then assessed in operative specimens of 251 patients who underwent total mesorectal excision (TME) without neoadjuvant radiotherapy in a multicenter clinical trial. Results: Methylation at MINT 2, 3, and 31 increased 11-fold (P=0.005), 15-fold (P<0.001), and 2-fold (P=0.02), respectively, during adenomatous transformation in normal rectal epithelium. Unsupervised grouping analyses of quantitative MINT methylation data of TME trial patients demonstrated two prognostic subclasses. In multivariate analysis of node-negative patients, this subclassification was the only predictor for distant recurrence (HR:4.17, 95% CI:1.72-10.10, P=0.002), cancer-specific survival (HR:3.74, 95% CI:1.48-9.43, P=0.003) and overall survival (HR:2.68, 95% CI:1.41-5.11, P=0.005). Conclusion: Methylation levels of specific MINT loci can be used as prognostic variables in patients with AJCC stage I and II rectal cancer. Quantitative epigenetic classification of rectal cancer merits evaluation as a stratification factor for adjuvant treatment in early disease.

### Introduction

In this study, we have focused specifically on epigenetic changes of rectal cancers from a clinical trial.

Epigenetic instability, such as changes in genomic DNA methylation status, is an early event during gastrointestinal tumor development and encompasses both hyper- and hypo-methylation changes.⁶⁻⁸ Most epigenetic cancer studies focus on specific genomic loci and analyze methylation status in a dichotomous manner, categorizing specimens as methylated or unmethylated. Also, in the majority of the studies assessing epigenetic changes and association with clinical outcome, non-quantitative measures are used, using a binary methylation status result. Absolute quantitative interpretation of methylation data would improve analysis of epigenetic events.⁹ Recently, we developed an assay for absolute quantitative assessment of methylated alleles (AQAMA) and showed quantitative methylation events to be associated with colorectal tumor progression.¹⁰ AQAMA measures the amount of methylated and unmethylated copy numbers simultaneously in a single reaction. The assay has excellent linearity in assessing DNA methylation levels and can be used on paraffin-embedded archival tissue (PEAT) sections treated with the on-slide (in situ) sodium bisulfite modification (SBM) technique that allows microdissected histology-oriented assessment of small (1-2 mm²) lesions.^{11,12} This allows efficient comparison of precursor adenoma and normal cells adjacent to tumor cells.

Methylation levels of methylated-in-tumor (MINT) loci have not been specifically tested for prognostic utility in rectal cancer. MINT loci are CpG-dinucleotide-rich regions located in non-protein-encoding DNA regions, and have been reported to become methylated in a tumor- and adenoma-specific manner in gastric and colon cancer.¹³⁻¹⁷ In a preliminary study, we quantified methylation levels of seven MINT loci at different stages of rectal tumor formation comparing paired normal-adenoma and adenoma-cancer tissues, and subsequently analyzed whether methylation level changes related to rectal tumor progression. We believed that methylation levels at MINT loci have prognostic significance for early rectal cancer progression. We then assessed the potential prognostic utility of MINT loci in primary tumor tissues from patients enrolled in a multicenter, randomized, surgical clinical trial. In this translational study analysis, unsupervised cluster analysis identified a subclass of patients whose quantitative methylation data were independently prognostic of progression to distant disease.

### Materials and Methods

Tissue specimens

In the preliminary study, patients operated on for rectal cancer with histopathologically confirmed adenocarcinoma were identified from the cancer registry database at SJHC. Only patients operated after 1995 were evaluated because of possible DNA degradation. Further selection of specimens was based on pathology-documented presence of tumor, as well as adenoma cells on the same tissue section.

For the clinical correlation studies, primary tumor PEAT specimens were obtained from 322 non-irradiated patients enrolled in the multi-center, randomized, quality-controlled TME trial coordinated by the Dutch Colorectal Cancer Group.³ The trial investigated whether neoadjuvant radiotherapy (5×5 Gy) before TME improved local control compared to TME surgery alone in patients with all stages of rectal cancer. Trial eligibility criteria and follow-up protocols have been described previously.^{3,18,19} All TME trial patients enrolled at the Dutch multicenter study sites were eligible further adhering to the following criteria: non-irradiated, TNM-stage I-III, with no evidence of disease (NED) after surgery. We opted to analyze the treatment arm because potential effects of radiation on genomic methylation are not known. Research protocols for the methylation studies on PEAT were approved by SJHC/JWCI and Leiden University Medical Center IRBs.

DNA Preparation from PEAT Specimens for Preliminary and Clinical Studies

From the preliminary study specimens, two consecutive sections (4 and 7 μm) of each PEAT block were cut and placed on adhesive-coated slides. The 4 μm section was stained with H&E and mounted. Tissue areas with normal epithelial, classic adenomatous, and invasive cancer cells were identified and marked off by an expert surgical pathologist. The tissue categories were histopathologically identified. Cancer cells were only taken from areas with nuclear atypia and signs of invasion of tissue architectural boundaries, the hallmark of cancer. Adenomatous cells were only taken from areas with classic villous and/or tubular adenomatous dysplasia. We did not include adenomatous tissue in the analysis with highly-dysplastic features without signs of invasion. The 7 μm section was treated by on-slide SBM as described previously.¹¹ Target tissue areas were identified and microdissected under a light microscope. Isolated cells were digested and 1 μl of the lysate was used for PCR.

From the clinical study TME trial patient specimens, tissue sections (7 μm) were cut from PEAT specimens and mounted on non-adhesive glass slides. Tumor-representative areas on H&E stained sections were marked by a surgical pathologist specializing in rectal cancer. Two sections per patient were deparaffinized, and the marked tissue was carefully microdissected. DNA was isolated and modified by sodium bisulfite, as previously described.²⁰ Salmon sperm DNA was added as a carrier.²¹ DsDNA and ssDNA were quantified before and after SBM by PicoGreen and OliGreen assays (Molecular Probes), respectively. Sufficient input DNA for AQAMA was determined as described.¹⁰ A salmon sperm DNA sample without tumor DNA was included in triplicate to assess background signal. Tissue blocks and isolated DNA were coded to prevent any bias.

### AQAMA MINT Locus Methylation Level Assessment

Absolute quantitative assessment of methylated alleles at MINT loci 1, 2, 12, and 31 has been described previously.¹⁰ Unpublished primer and probe sets for the remaining three MINT loci were: MINT3, 5′-TGATGGTGTATGTGATTTTGTGTT-3′ (forward), 5′-ACCCCACCCCTCACAAAC-3′ (reverse), 5′-ACCTACGAACGAACAC- 3′ (methylated probe), 5′-TACCTACAAACAAACAC-3′ (unmethylated probe); MINT17, 5′-AGGGGTTAGGTTGAGGTTGTT-3′ (forward), 5′-TCTACCTCTTCCCAAATTCCA-3′ (reverse), 5′-TTGGATGGATCGCGG- 3′ (methylated probe), 5′-TATTTTGGATGGATTGTGG-3′ (unmethylated probe); MINT25, 5′-GGGGATAGGAAGATGGTTT-3′ (forward), 5′-CCCCCATCCCATACAACC-3′ (reverse), 5′-TTTGTTTCGTAGCGGAGT-3′ (methylated probe), 5′-GATTTTGTTTTGTAGTGGAG-3′ (unmethylated probe). DNA samples were run in 384-well microplates in triplicate, and each plate contained individual marker cDNA standards with known copy numbers, allowing assessment of absolute methylated and unmethylated copy number. Controls for specificity of AQAMA for methylated and unmethylated sequences, as well as controls for non-specific amplification, were included.^{10,22} Final analysis outcome was the methylation index (MI), calculated as: [copy number_{methylated alleles}/(copy number_{methylated alleles}+copy number_{unmethylated alleles})].

### Profiling by Unsupervised Random Forest Clustering

For identification of patient clusters with similar MINT locus methylation profiles, we employed unsupervised random forest (RF) clustering²³, as it has been successfully applied in comparable data sets (Supplemental Appendix 1).^{24,25}

### Results

### MINT Locus Methylation Levels During Rectal Cancer Development

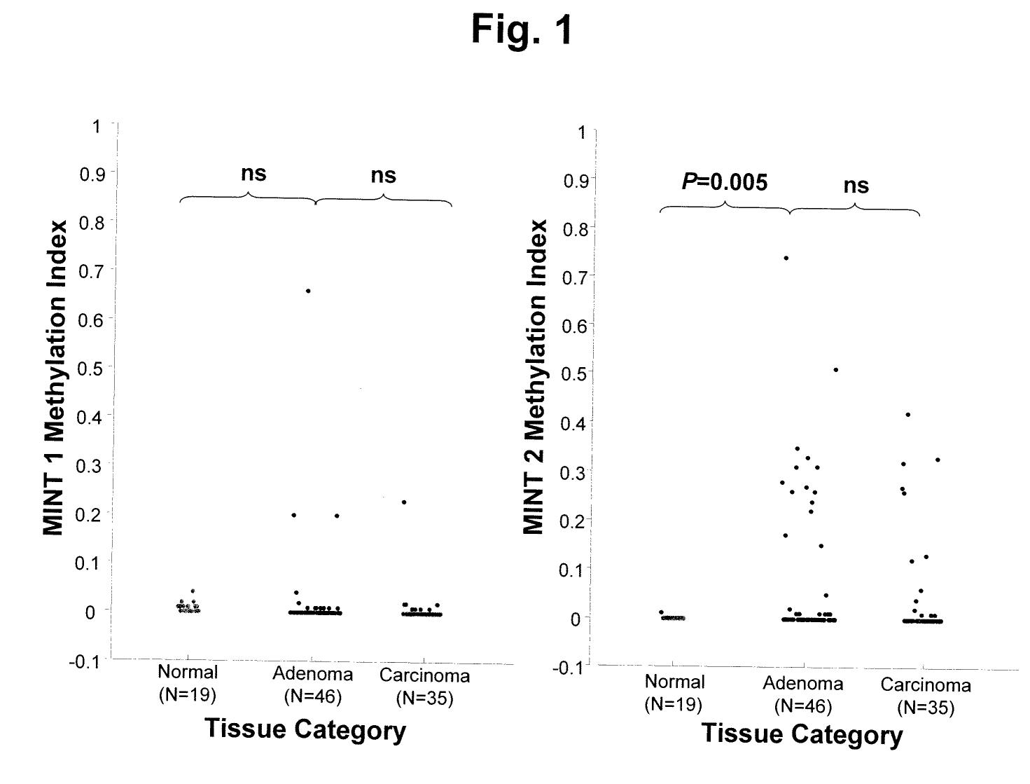

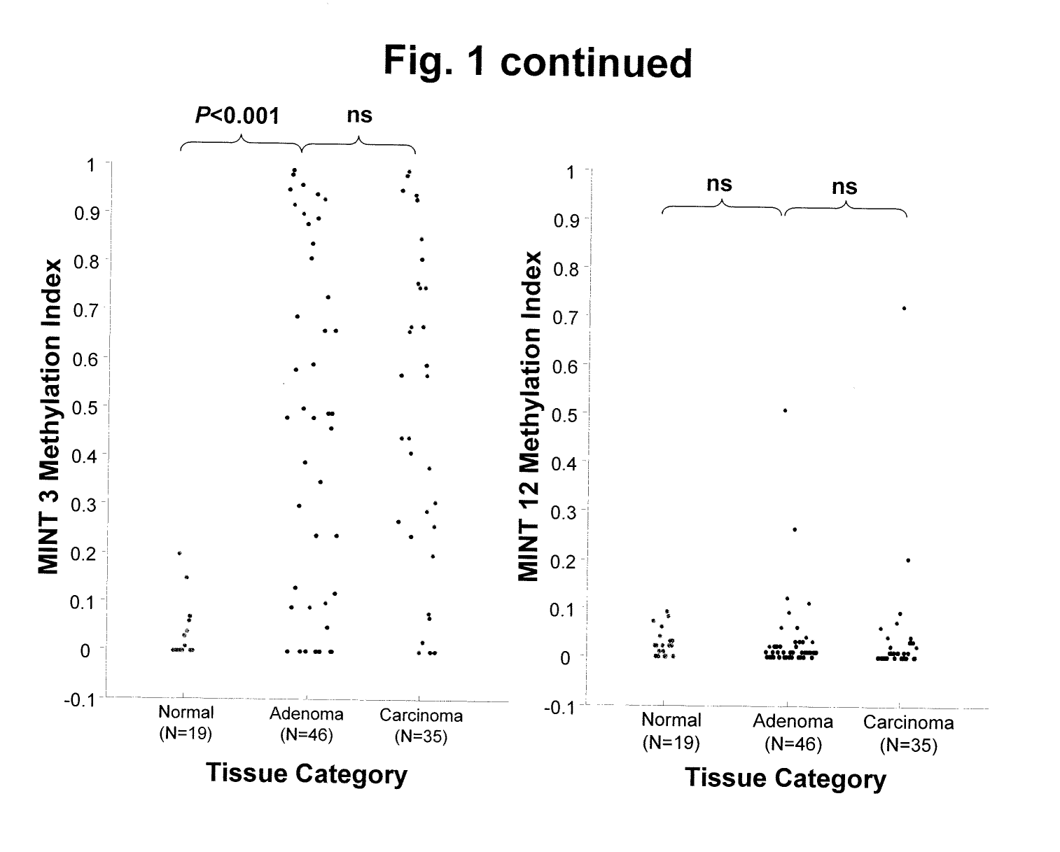

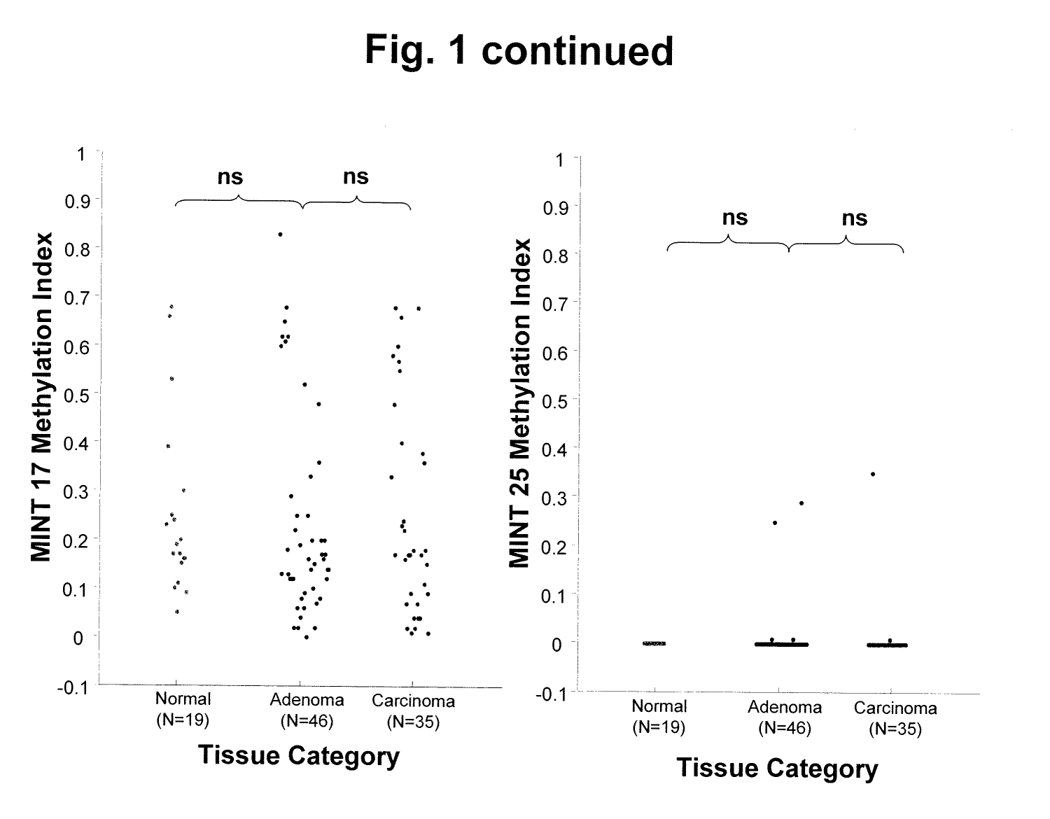

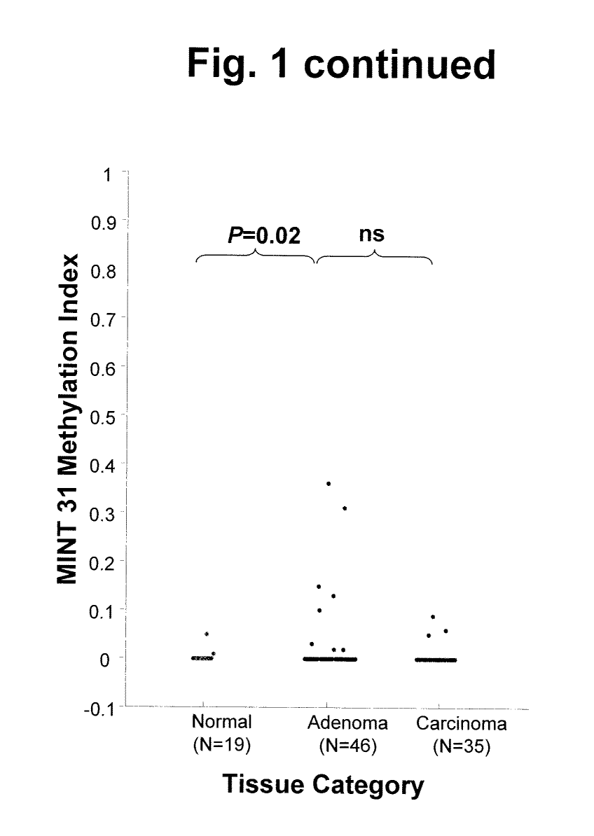

Sets of normal, adenomatous, and malignant PEAT tissues from 46 patients with rectal cancer were examined by AQAMA of MINT loci known to be differentially methylated in colorectal cancer.¹² The H&E-stained sections cut from the tissue blocks that, according to the diagnostic pathology report, contained adenoma as well as cancer tissue, were histopathology-evaluated by an expert pathologist. In the 46 tissue sections, 19, 46, and 35 areas of normal epithelium, adenoma, and cancer tissue, respectively, were identified. This resulted into paired analyses of 19 normal-adenoma sets and 35 adenoma-cancer sets. FIG. 1 shows scatterplots of the MI values in the three histopathology categories for each MINT locus. MINT loci 2, 3, and 31 underwent a significant increase in absolute mean methylation level during adenomatous transformation. There were no significant MINT methylation changes for any MINT locus during progression from adenoma to cancer. Subsequently, the significant increases were early events associated with dysplastic change of normal rectal epithelium. Because three MINT loci (2, 3, 31) showed significant increase in methylation levels and the normal distribution of the quantitative methylation data sets in healthy rectal epithelium changes to non-normal in adenoma in four other loci (1, 12, 17, 25) (Supplemental Appendix 2), all seven MINT loci were considered to have potential utility to identify epigenetic subclasses in the clinical study patient group.

### Sample Size Calculations

To establish the sample size for the clinical study, we performed power calculations using methylation results of the preliminary study and recurrence rates of the TME trial. It was calculated that 250 patients were sufficient to obtain significance for predicting distant recurrence with an alpha-level of 0.05 and 90% power. Because the available patient specimens from the trial were primary tumor PEAT blocks from various hospital sites, we allowed for 30% loss of cases due to availability and quality of tissue and DNA. We therefore required 72 additional cases, and the final sample size was set at 325 cases. 672 patients fulfilled our study criteria (see Material and Methods). Finally, of 314 cases, DNA was isolated (in 11 cases, tumor cell number was insufficient). Subsequently, of the 314 DNA isolations, after processing and bisulfite treatment, only 251 had sufficient input DNA for AQAMA. Characteristics of the 251 patients finally analyzed were not significantly different in prognostic factors and characteristics from the original trial population (Supplemental Appendix 3).

### MINT Locus Methylation Profile Identification

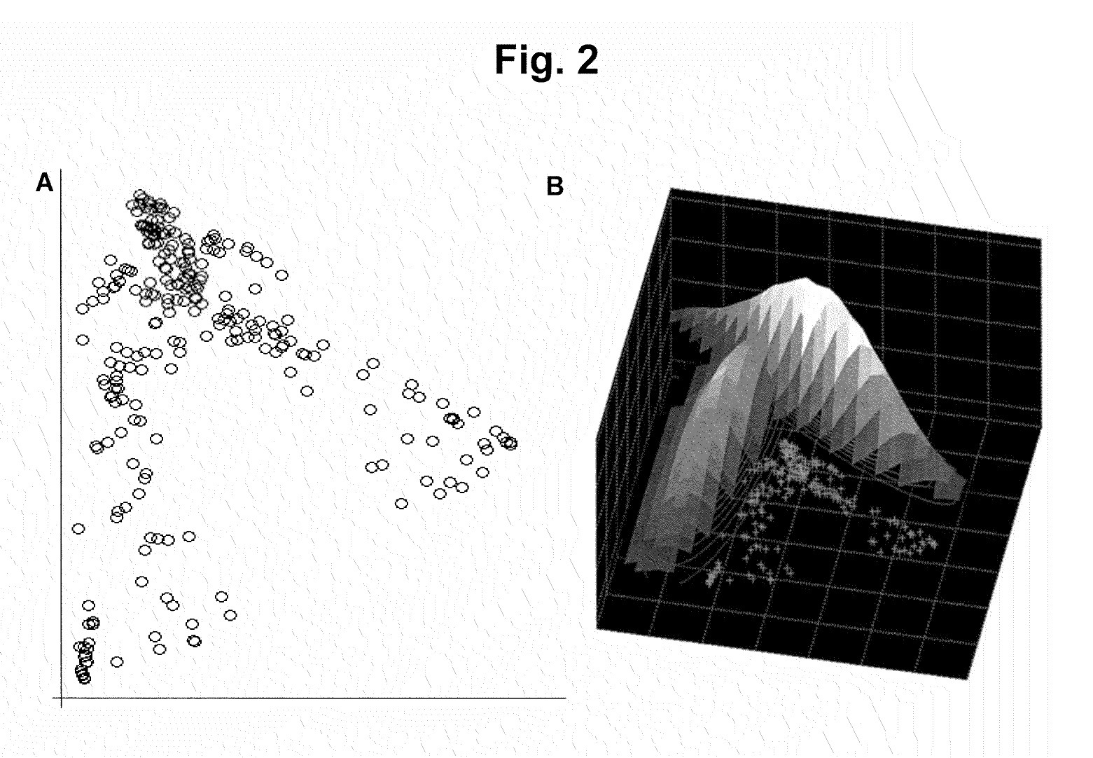

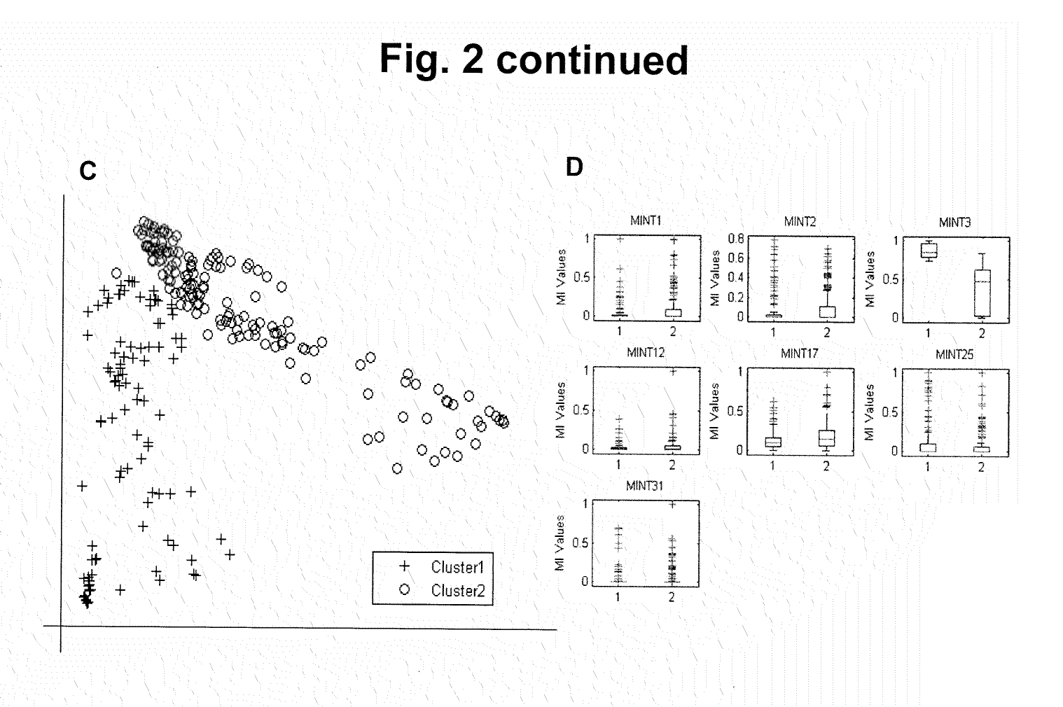

To investigate whether rectal cancer can be grouped by methylation level at specific MINT loci, we performed unsupervised RF clustering on the quantitative methylation level results of patients from the TME trial. As an outcome, a MDS plot indicated the mutual distance between the cases based on methylation level of all seven MINT loci (FIG. 2A). Inspection of the MDS plot indicated two groups of rectal cancer cases. To identify which patients belonged to which group, we performed an EM-MoG analysis based on the Gaussian shape of patient clusters (FIG. 2B,C). The EM-MoG algorithm allocated the patients based on the likelihood to fall under the normal (Gaussian) distribution of one of the two clusters. Subsequently, variable importance and the methylation patterns matching the identified clusters were analyzed (FIG. 2D, Table 1A). The 89 patients (35%) allocated to cluster 1 had significantly increased methylation at MINT3 and significantly decreased methylation at MINT1, 12, and 17 as compared with patients in cluster 2. The unsupervised clustering results showed that subclasses of rectal cancers can be identified by differences in DNA methylation level of tested MINT loci. The Gini-index indicated that MINT3 and MINT17 were the most important variables in forming the clusters.

TABLE 1A
Variable Importance by Gini Index and Comparison of
Mean MINT Locus MI Values Between Identified Clusters

| | | All patients | | | Node-negative patients | | |
|---|---|---|---|---|---|---|---|
| | | (n = 251) | | | (n = 145) | | |
| | | Cluster 1 | Cluster 2 | | Cluster 1 | Cluster 2 | |
| | Gini | (n = 89) | (n = 162) | | (n = 55) | (n = 90) | |
| MINT locus | Index | Median^{†} | Median^{†} | P-Value* | Median^{†} | Median^{†} | P-Value* |
| MINT1 | 11.6 | 0.00 | 0.01 | <0.001 | 0.00 | 0.00 | 0.006 |
| | | (0.00-0.01) | (0.00-0.09) | | (0.00-0.02) | (0.00-0.09) | |
| MINT2 | 10.8 | 0.08 | 0.00 | 0.07 | 0.00 | 0.00 | 0.51 |
| | | (0.00-0.02) | (0.00-0.12) | | (0.00-0.03) | (0.00-0.10) | |
| MINT3 | 20.2 | 0.87 | 0.50 | <0.001 | 0.84 | 0.49 | <0.001 |
| | | (0.79-0.99) | (0.06-0.65) | | (0.79-0.99) | (0.06-0.65) | |
| MINT12 | 13.5 | 0.03 | 0.02 | 0.01 | 0.02 | 0.02 | 0.22 |
| | | (0.00-0.02) | (0.01-0.05) | | (0.00-0.02) | (0.00-0.05) | |
| MINT17 | 20.7 | 0.08 | 0.21 | <0.001 | 0.09 | 0.20 | 0.005 |
| | | (0.04-0.13) | (0.08-0.30) | | (0.05-0.15) | (0.12-0.24) | |
| MINT25 | 12.1 | 0.00 | 0.00 | 0.21 | 0.00 | 0.00 | 0.81 |
| | | (0.00-0.04) | (0.00-0.08) | | (0.00-0.05) | (0.00-0.09) | |
| MINT31 | 6.0 | 0.00 | 0.00 | 0.90 | 0.00 | 0.00 | 0.82 |
| | | (0.00-0.00) | (0.00-0.00) | | (0.00-0.00) | (0.00-0.00) | |

^{†}Interquartile range
*Calculated by Mann-Whitney's u-test

### Clinicopathological Correlation and Distant Recurrence Analyses

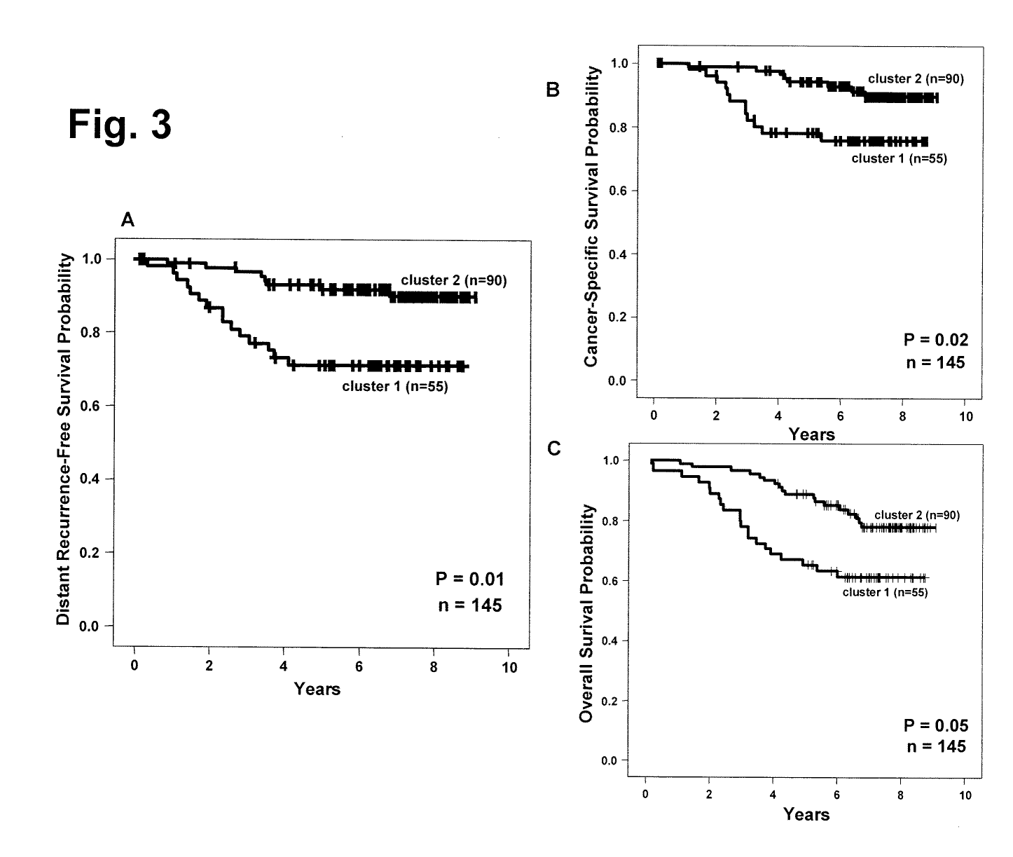

There were no significant associations observed in epigenetic subclasses of rectal cancer to any of the investigated standard clinical or tumor-pathological factors (Table 1B). The preliminary results demonstrated that methylation level differences at the specific MINT loci develop early during tumor formation. There was no significant relation between cluster allocation and clinico-pathological factors in node-negative tumors (Table 1B). Because identification of stage I and II patients at risk for distant metastasis is highly clinically relevant and there was no dependence of the identified patient clusters to nodal status we excluded stage III patients from distant disease recurrence analyses. We assessed the probability of distant disease recurrence, cancer-specific, and overall survival (OS). Because EM-MoG analysis is a probability-based cluster assignment algorithm, we performed multiple imputation analysis to correct for cases that have a small difference in probability to be assigned to either one of the clusters. In node-negative patients, cluster 1 patients had significant increased risk for distant recurrence (P=0.01), shorter cancer-specific survival (P=0.02), and shorter OS (P=0.05, FIG. 3A-C). At the time of the analyses, median duration of follow-up was 7.1 years (range 2.5-9.8 years).

TABLE 1B
Comparison of Clinical and Tumor Pathology Factors
and MINT Locus Clusters

| | All Patients | | | Node-negative Patients | | |
|---|---|---|---|---|---|---|
| | (n = 251) | | | (145) | | |
| Clinical and Tumor | Cluster 1 | Cluster 2 | | Cluster 1 | Cluster 2 | |
| Pathology Factors | n = 89 | n = 162 | P-value | n = 55 | n = 90 | P-value |
| Sex | | | | | | |
| Male | 64 (39) | 98 (61) | 0.08 | 38 (40) | 56 (60) | 0.40 |
| Female | 25 (28) | 64 (72) | | 17 (33) | 34 (67) | |
| Age | | | | | | |
| Mean (SE) | 64.8 (1.2) | 62.5 (0.9) | 0.15 | 65.4 (1.7) | 63.4 (1.2) | 0.33 |
| TNM-stage | | | | | | |
| 1 | 29 (41) | 41 (59) | 0.22 | 29 (41) | 41 (59) | 0.40 |
| II | 26 (35) | 49 (65) | | 26 (35) | 49 (65) | |
| III | 34 (32) | 72 (68) | | — | — | |
| N-status | | | | | | |
| N₀ (≧12 examined) | 12 (35) | 22 (65) | 0.53 | 12 (35) | 22 (65) | 0.84 |
| N₀/Nₓ (<12 examined) | 44 (39) | 68 (61) | | 43 (39) | 68 (61) | |
| N₁ (1-3 positive) | 21 (34) | 40 (66) | | — | — | |
| N₂ (≧4 positive) | 12 (27) | 32 (73) | | — | — | |
| Differentiation | | | | | | |
| Well | 5 (28) | 13 (72) | 0.78 | 3 (33) | 6 (67) | 0.99 |
| Moderately | 66 (37) | 110 (63) | | 44 (39) | 70 (61) | |
| Poor | 18 (32) | 39 (68) | | 8 (36) | 14 (64) | |
| Location distant recurrences | | | | | | |
| Liver | 11 (41) | 16 (59) | 0.62 | 4 (50) | 4 (50) | 0.37 |
| Non-liver | 20 (49) | 21 (51) | | 11 (73) | 4 (27) | |
| Resection type | | | | | | |
| Low anterior | 54 (32) | 113 (68) | 0.26 | 32 (34) | 62 (66) | 0.35 |
| Abdominoperineal | 33 (43) | 44 (57) | | 21 (47) | 24 (53) | |
| Hartmann | 2 (29) | 5 (71) | | 2 (33) | 4 (67) | |
| Circumferential margin | | | | | | |
| Negative | 72 (35) | 131 (65) | 0.39 | 52 (39) | 81 (61) | 0.54 |
| Positive | 17 (35) | 31 (65) | | 3 (25) | 9 (75) | |

### Multivariate Analyses

Multivariate analyses were performed to assess whether the observed prognostic value of the clusters was independent from standard prognostic variables for the complete patient group and for node-positive and negative patients (Table 2). T-stage, N-stage, circumferential margin status, distance of the tumor to the anal verge, and tumor differentiation were considered in a Cox's regression analysis. In node-negative patients, the quantitative MINT locus methylation profile was, of the considered variables, the only selected predictive factor for distant disease recurrence and cancer-specific survival. OS was also affected by T-stage in patients without nodal involvement. Circumferential margin involvement of the tumor and short (<5 cm) distance of the tumor from the anal verge increased the risk of distant recurrence and decreased cancer-specific survival and OS in node-positive rectal cancer patients. Possible dependence of the results on any of the 42 different study sites was evaluated in the published clinical trial report²⁶ and ruled out also in our analyses. The multivariate results show that the identified subclass of rectal cancers is independently predictive of distant recurrence.

TABLE 2
Multivariate Analysis Results of All Patients and Node-negative Patients

| | All patients | | Node-negative | | Node-positive | |
|---|---|---|---|---|---|---|
| | n = 251 | | n = 145 | | n = 106 | |
| | HR | | HR | | HR | |
| Variable | (95% CI) | P-value | (95% CI) | P-value | (95% CI) | P-value |

Distant recurrence

| | | | | | | |
|---|---|---|---|---|---|---|
| T-stage (3/4) | 1.70 | 0.09 | 1.19 | 0.70 | 2.91 | 0.05 |
| | (0.92-3.16) | | (0.49-2.93) | | (1.01-8.37) | |
| Nodal status (+) | 2.47 | 0.001 | — | — | — | ns |
| | (1.44-4.23) | | | | | |
| Circumferential margin (+) | 1.87 | 0.03 | 2.40 | 0.21 | 1.77 | 0.08 |
| | (1.07-3.28) | | (0.62-9.39) | | (0.94-3.33) | |
| Distance from anal verge >5 cm | 0.71 | 0.19 | 1.50 | 0.40 | 0.50 | 0.03 |
| | (0.43-1.18) | | (0.59-3.85) | | (0.27-0.92) | |
| Poor differentiation | 1.39 | 0.23 | 1.24 | 0.71 | 1.59 | 0.16 |
| | (0.81-2.38) | | (0.40-3.89) | | (0.84-3.01) | |
| MINT locus profile (cluster 1) | 1.68 | 0.04 | 4.17 | 0.002 | 1.11 | 0.75 |
| | (1.03-2.73) | | (1.72-10.10) | | (0.59-2.09) | |

Cancer-specific survival

| | | | | | | |
|---|---|---|---|---|---|---|
| T-stage (3/4) | 2.12 | 0.03 | 1.88 | 0.21 | 2.85 | 0.05 |
| | (1.07-4.19) | | (0.70-5.04) | | (0.98-8.26) | |
| Nodal status (+) | 2.47 | 0.002 | — | — | — | ns |
| | (1.41-4.35) | | | | | |
| Circumferential margin (+) | 1.93 | 0.02 | 2.28 | 0.23 | 1.88 | 0.05 |
| | (1.09-3.41) | | (0.59-8.81) | | (0.99-3.56) | |
| Distance from anal verge >5 cm | 0.59 | 0.05 | 1.46 | 0.46 | 0.40 | 0.004 |
| | (0.35-0.99) | | (0.53-4.03) | | (0.22-0.75) | |
| Poor differentiation | 1.56 | 0.11 | 1.28 | 0.67 | 1.70 | 0.12 |
| | (0.91-2.70) | | (0.41-4.06) | | (0.88-3.29) | |
| MINT locus profile (cluster 1) | 1.47 | 0.15 | 3.74 | 0.005 | 0.99 | 0.98 |
| | (0.88-2.45) | | (1.48-9.43) | | (0.51-1.93) | |

Overall survival

| | | | | | | |
|---|---|---|---|---|---|---|
| T-stage (3/4) | 1.92 | 0.01 | 2.12 | 0.04 | 1.98 | 0.10 |
| | (1.14-3.23) | | (1.05-4.29) | | (0.87-4.50 | |
| Nodal status (+) | 1.88 | 0.004 | — | ns | — | ns |
| | (1.22-2.92) | | | | | |
| Circumferential margin (+) | 1.66 | 0.04 | 1.65 | 0.33 | 1.66 | 0.08 |
| | (1.02-2.69) | | (0.60-4.53) | | (0.95-2.91) | |
| Distance from anal verge >5 cm | 0.69 | 0.09 | 0.96 | 0.92 | 0.55 | 0.03 |
| | (0.45-1.06) | | (0.49-1.90) | | (0.32-0.95) | |
| Poor differentiation | 1.33 | 0.22 | 1.06 | 0.90 | 1.37 | 0.28 |
| | (0.84-2.09) | | (0.45-2.47) | | (0.77-2.44) | |
| MINT locus profile (cluster 1) | 1.48 | 0.06 | 2.68 | 0.003 | 1.00 | 1.00 |
| | (0.98-2.24) | | (1.41-5.11) | | (0.57-1.77) | |

### MINT3 and MINT17

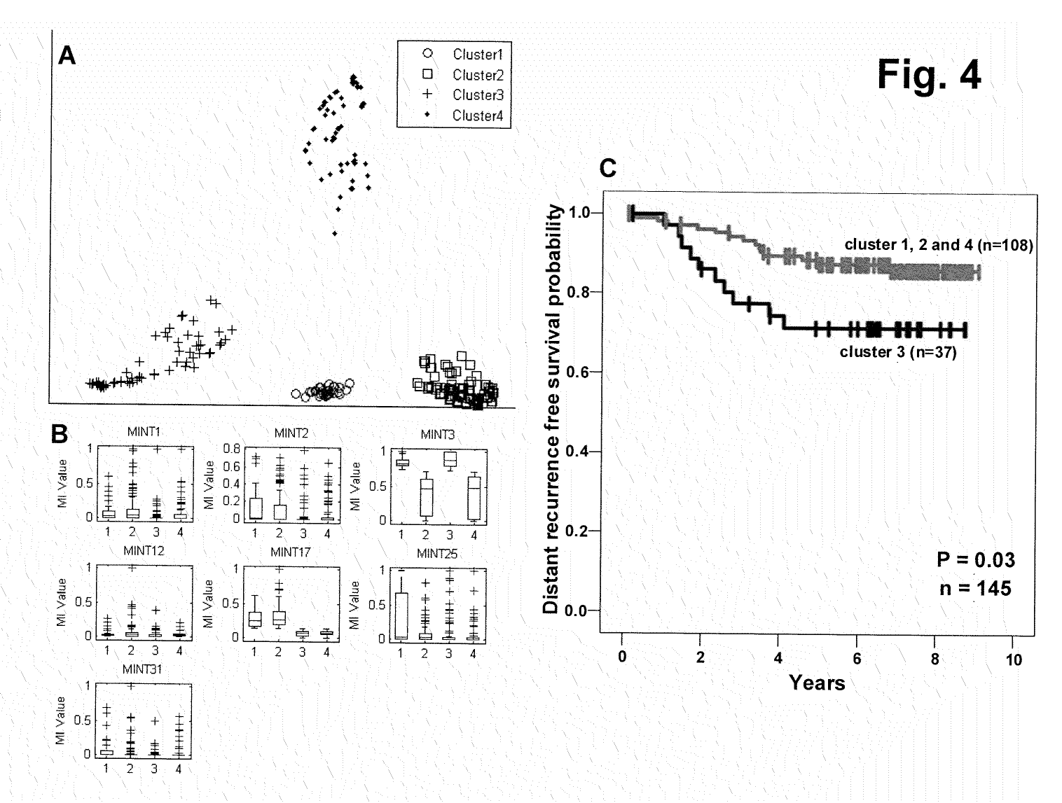

The Gini-index indicating variable importance in RF clustering shown in Table 1A demonstrated MINT3 and MINT17 to hold the most information to form the two clusters compared to the other five MINT loci. We continued to assess whether methylation levels at MINT3 and MINT17 have prognostic value as a separate marker set. The quantitative methylation data of MINT3 and MINT17 were entered into the RF algorithm and the resulting MDS plot is given in FIG. 4A. Four clearly separate clusters are formed and the corresponding methylation level differences between the clusters are plotted in FIG. 4B. Cluster 3 containing 67 patients (27%) corresponds to the previously identified high-risk cluster 1 as average MINT3 methylation index are relatively high and MINT17 methylation index is relatively low. In KM analysis, cluster 3 patients are shown to be at significantly increased risk for distant metastasis in node-negative patients compared to the other three clusters (FIG. 4C). In multivariate analysis the results showed that the high-risk cluster 3 was selected as the only independent factor among the variables analyzed predicting in node-negative patients distant recurrence probability (HR:2.84, 95% CI:1.22-6.62, P=-0.02), cancer-specific (HR:3.29, 95% CI:1.33-8.12, P=0.01) and overall survival (HR:2.21, 95% CI:1.13-4.29, P=0.02). It was concluded that patients at increased risk for distant metastasis can be defined as having tumors with a MINT3 methylation level>0.72 and MINT17 methylation level<0.14. The analysis also demonstrated that the specific combination of increased methylation at MINT3 and decreased methylation at MINT17 is required for the prognostic information.

### Discussion

Most studies of biomarkers in large bowel adenocarcinoma include colon, as well as rectum, even though rectal and colon cancers are treated differently. Moreover, right-sided and left-sided bowel adenocarcinomas have different molecular patterns; microsatellite instability and methylator phenotype are rarely seen in the rectum.²⁷ Our data represents one of the largest clinical analyses of methylation biomarkers in rectal cancer specifically, and also demonstrates the first quantitative correlation between MINT methylation levels and disease progression.

The preliminary study demonstrated a progressive increase in methylation levels of specific MINT loci comparing normal and adenomatous rectal tissue. No significant change in methylation level at any MINT locus was detected comparing adenomatous and malignant rectal tissue. A correlation between methylation of MINT loci and development of adenomatous dysplasia has been reported.¹⁷ Our data are unique, as we used paired normal-adenoma-cancer specimens, quantitative techniques, and analyzed rectal cancers only. The results of our clinical study identified two prognostic categories of rectal cancer based strictly on the absolute quantitative differences in methylation level. Our data show that methylation levels at multiple and two specific identified MINT loci are related to rectal tumor formation, and that they may be seen as surrogate markers of distant rectal cancer disease recurrence and disease survival. The role of non-coding regions have been of much interest in that they may be influential in gene encoding regions.²⁸⁻³⁰ Especially interesting is that the chromosomal location (1p36) of the MINT3 locus, which undergoes methylation in most rectal adenomas, contains many cancer-related genes. Methylation of MINT loci 1, 2, 12, and 31 is often studied in relation to the CpG island methylator phenotype (CIMP) that forms a subclass of right colon tumors closely associated with microsatellite instability (MSI).³¹ In our study, the unsupervised clustering analyses did not identify a CIMP associated with hypermethylation in the selected MINT loci. Interestingly, a combination of relative hyper- as well as hypo-methylation was observed in the identified subclasses. This specific combination was even required to show prognostic value on rectal cancer distant recurrence rates. This corroborates that CIMP does not occur in the rectum and that rectal cancer may have different epigenetic pathological changes compared to proximal colon adenocarcinoma. Reported correlations between MINT 1, 2, 12, and 31 and clinico-pathological features overlap with the features of MSI(+) tumors (right-sidedness, poor differentiation, early stage) and therefore our results can not be compared.^{27,32,33} We previously showed relevance of the AQAMA technique testing methylation levels at MINT 1, 2, 12, and 31 and increased methylation at this loci detected by the AQAMA assay was significantly correlated to right-sided colon tumors.¹⁰

Our preliminary study data indicates that methylation events at the measured MINT loci are related to early dysplastic proliferation of subclasses of rectal premalignancies and MINT loci may be a clinical biomarker. Subsequently, in a large rectal cancer patient group, RF clustering was able to identify, in an unbiased manner, two groups of rectal cancer patients that were naturally present within the quantitative methylation data. This demonstrated that subclassification of rectal cancer patients can be made based on absolute quantitative methylation level differences.

There was no correlation between MINT methylation profile and nodal status; in node-negative patients, the MINT profile was the only selected variable in multivariate analyses for distant recurrence probability and, subsequently, for cancer-specific survival. Identifying stage I and II patients at risk for distant disease recurrence, assessing primary tumors for predictive genomic biomarkers would be important for stratifying adjuvant treatment. Moreover, since accurate upstaging from stage II to III remains a difficult task,³⁴ we approached this by a quantitative analysis of a specific panel of epigenetic biomarkers. The advantage of using genomic analysis is the stability of DNA as compared to mRNA in PEAT, where, in the latter, there is a higher level of degradation with time.

### REFERENCES

1. Jemal A, Siegel R, Ward E, et al: Cancer statistics, 2007. CA Cancer J Clin 57:43-66, 2007

2. Improved survival with preoperative radiotherapy in resectable rectal cancer. Swedish Rectal Cancer Trial. N Engl J Med 336:980-7, 1997

3. Kapiteijn E, Marijnen C A, Nagtegaal I D, et al: Preoperative radiotherapy combined with total mesorectal excision for resectable rectal cancer. N Engl J Med 345:638-46, 2001

4. Bosset J F, Collette L, Calais G, et al: Chemotherapy with preoperative radiotherapy in rectal cancer. N Engl J Med 355:1114-23, 2006

5. Iacopetta B: Are there two sides to colorectal cancer? Int J Cancer 101:403-8, 2002

6. Jones P A, Gonzalgo M L: Altered DNA methylation and genome instability: a new pathway to cancer? Proc Natl Acad Sci USA 94:2103-5, 1997

7. Jones P A, Baylin S B: The fundamental role of epigenetic events in cancer. Nat Rev Genet 3:415-28, 2002

8. Baylin S B, Herman J G: DNA hypermethylation in tumorigenesis: epigenetics joins genetics. Trends Genet 16:168-74, 2000

9. Zeschnigk M, Bohringer S, Price E A, et al: A novel real-time PCR assay for quantitative analysis of methylated alleles (QAMA): analysis of the retinoblastoma locus. Nucleic Acids Res 32:e125, 2004

10. de Maat M F, Umetani N, Sunami E, et al: Assessment of methylation events during colorectal tumor progression by absolute quantitative analysis of methylated alleles. Mol Cancer Res 5:461-71, 2007

11. Umetani N, de Maat M F, Sunami E, et al: Methylation of p16 and Ras association domain family protein 1a during colorectal malignant transformation. Mol Cancer Res 4:303-9, 2006

12. Toyota M, Ho C, Ahuja N, et al: Identification of differentially methylated sequences in colorectal cancer by methylated CpG island amplification. Cancer Res 59:2307-12, 1999

13. Issa J P, Shen L, Toyota M: CIMP, at last. Gastroenterology 129:1121-4, 2005

14. Toyota M, Ahuja N, Ohe-Toyota M, et al: CpG island methylator phenotype in colorectal cancer. Proc Natl Acad Sci USA 96:8681-6, 1999

15. Toyota M, Ohe-Toyota M, Ahuja N, et al: Distinct genetic profiles in colorectal tumors with or without the CpG island methylator phenotype. Proc Natl Acad Sci USA 97:710-5, 2000

16. Toyota M, Ahuja N, Suzuki H, et al: Aberrant methylation in gastric cancer associated with the CpG island methylator phenotype. Cancer Res 59:5438-42, 1999

17. Wynter C V, Kambara T, Walsh M D, et al: DNA methylation patterns in adenomas from FAP, multiple adenoma and sporadic colorectal carcinoma patients. Int J Cancer 118:907-15, 2006

18. Peeters K C, Kapiteijn E, van de Velde C J: Managing rectal cancer: the Dutch experience. Colorectal Dis 5:423-6, 2003

19. Kapiteijn E, van de Velde C J: Developments and quality assurance in rectal cancer surgery. Eur J Cancer 38:919-36, 2002

20. Spugnardi M, Tommasi S, Dammann R, et al: Epigenetic inactivation of RAS association domain family protein 1 (RASSF1A) in malignant cutaneous melanoma. Cancer Res 63:1639-43, 2003

21. Herman J G, Graff J R, Myohanen S, et al: Methylation-specific PCR: a novel PCR assay for methylation status of CpG islands. Proc Natl Acad Sci USA 93:9821-6, 1996

22. Umetani N, de Maat M F, Mori T, et al: Synthesis of universal unmethylated control DNA by nested whole genome amplification with phi29 DNA polymerase. Biochem Biophys Res Commun 329:219-23, 2005

23. Liaw A, Wiener M: Classification and Regression by Random Forest. R News 2/3:18-22, 2002

24. Seligson D B, Horvath S, Shi T, et al: Global histone modification patterns predict risk of prostate cancer recurrence. Nature 435:1262-6, 2005

25. Shi T, Seligson D, Belldegrun A S, et al: Tumor classification by tissue microarray profiling: random forest clustering applied to renal cell carcinoma. Mod Pathol 18:547-57, 2005

26. Kapiteijn E, Kranenbarg E K, Steup W H, et al: Total mesorectal excision (TME) with or without preoperative radiotherapy in the treatment of primary rectal cancer. Prospective randomised trial with standard operative and histopathological techniques. Dutch ColoRectal Cancer Group. Eur J Surg 165:410-20, 1999

27. van Rijnsoever M, Grieu F, Elsaleh H, et al: Characterisation of colorectal cancers showing hypermethylation at multiple CpG islands. Gut 51:797-802, 2002

28. Willingham A T, Gingeras T R: TUF love for “junk” DNA. Cell 125:1215-20, 2006

29. Wang J, Gonzalez K D, Scaringe W A, et al: Evidence for mutation showers. Proc Natl Acad Sci USA 104:8403-8, 2007

30. Lin S L, Ying S Y: Gene silencing in vitro and in vivo using intronic microRNAs. Methods Mol Biol 342:295-312, 2006

31. Weisenberger D J, Siegmund K D, Campan M, et al: CpG island methylator phenotype underlies sporadic microsatellite instability and is tightly associated with BRAF mutation in colorectal cancer. Nat Genet 38:787-93, 2006

32. Ward R L, Cheong K, Ku S L, et al: Adverse prognostic effect of methylation in colorectal cancer is reversed by microsatellite instability. J Clin Oncol 21:3729-36, 2003

33. Hawkins N, Norrie M, Cheong K, et al: CpG island methylation in sporadic colorectal cancers and its relationship to microsatellite instability. Gastroenterology 122:1376-87, 2002

34. Bilchik A J, Nora D T, Sobin L H, et al: Effect of lymphatic mapping on the new tumor-node-metastasis classification for colorectal cancer. J Clin Oncol 21:668-72, 2003

### Supplemental Appendix 1

### Profiling by Unsupervised Random Forest Clustering

Each tree is trained on a bootstrap dataset, drawn with replacement from the original dataset, and on a fixed number of randomly chosen predictors. Each tree, containing about two-third of the original dataset, places a vote on bagged data points. Bagging eliminates the need for a separate training and validation set in decision tree learning.¹ The multidimensional distances are expressed in the resulting proximity matrix of the RF algorithm. A series of 5000 trees were trained with 3 random variables per node. The random forest (RF) dissimilarity algorithm used is based on individual decision tree predictors and automatically dichotomizes the expressions into clusters in a data-driven approach. The groups were therefore not established by employing cut-offs. The Gini-index indicating individual MINT loci variable importance was calculated.²⁻⁴

### Cluster Assignment

We used the proximity matrix obtained from the RF clustering algorithm (represented in a multi-dimensional scaling (MDS)) plot to perform a cluster analysis.⁵ Cluster assignments were obtained by performing an expectation maximization algorithm with a mixture of Gaussians (EM-MoG) for two clusters on the scaled data. EM-MoG clustering is based on a two-step approach to fit Gaussian probability models on the data, in order to estimate most likely clusters. All analyses to identify the MINT locus profiles were performed using MATLAB software (v7.3, MathWorks) and “R” (see the website cran.r-project.org). The number of clusters was established by inspection of the MDS-plot.^{6,7} The posterior cluster probabilities are subsequently used in a multiple imputation procedure to account for cluster membership uncertainty.

### Further Statistical Analyses

In the preliminary study, differences between methylation levels of the normal, adenomatous, and cancerous tissue from the same PEAT block were assessed by non-parametric, Wilcoxon's rank sum-test for paired samples.

For the clinical study on TME trial patients, differences in survival, clinical and tumor-pathological factors between patients assigned by RF clustering were analyzed. Specimens that did not yield sufficient DNA quantity or quality for PCR were excluded. Chi-square tests were used to compare proportions. Mann-Whitney or Kruskal-Wallis u-tests were used to compare ordinal variables. Student's t-test was used to assess differences in age. Survival differences between groups were visualized by the Kaplan-Meier method and log-rank test assessed significance. The Cox proportional hazards model was used for multivariate analysis of time-to-event endpoints. Results are presented as hazard ratios and 95% confidence intervals. Co-variables entered in the model included T-stage, N-stage, circumferential margin status, distance of the tumor from the anal verge, and tumor differentiation. A two-sided P-value of 0.05 or less indicated statistical significance. All clinical correlative analyses with identified clusters were performed using SPSS statistical software (v12.0.1, SPSS Inc, Chicago). The day 0 point for the analyses of survival and recurrence was the day of surgery. Data on patients who were alive or free of recurrence were censored at the time of the last follow-up. Uncertainty in RF cluster membership assignment was taken into account using multiple imputation,⁸ where multiple (M=5) complete datasets were generated using the posterior cluster membership probabilities obtained from the EM-MoG algorithm.

### REFERENCES

1. Breiman L: Bagging Predictors, Machine Learning, 1996, pp 123-40

2. Qi Y, Bar-Joseph Z, Klein-Seetharaman J: Evaluation of different biological data and computational classification methods for use in protein interaction prediction. Proteins 63:490-500, 2006

3. Strobl C, Boulesteix A L, Zeileis A, et al: Bias in random forest variable importance measures: illustrations, sources and a solution. BMC Bioinformatics 8:25, 2007

4. Guyon I, Elisseeff A: An introduction to variable and feature selection. J Machine Learn Res 5:1157-82, 2003

5. Liaw A, Wiener M: Classification and Regression by Random Forest. R News 2/3:18-22, 2002

6. Seligson D B, Horvath S, Shi T, et al: Global histone modification patterns predict risk of prostate cancer recurrence. Nature 435:1262-6, 2005

7. Shi T, Seligson D, Belldegrun A S, et al: Tumor classification by tissue microarray profiling: random forest clustering applied to renal cell carcinoma. Mod Pathol 18:547-57, 2005

8. Little R J A, Rubin D B: Statistical Analysis with Missing Data. New York, Wiley, 2002

### Supplemental Appendix 2

The distribution of the methylation data for each MINT locus for each tissue category was characterized by Kurtosis and Kolmogorov-Smirnov (K-S) analysis testing adherence to the normal distribution assumption. We have previously used this approach in the context of quantitative methylation data.¹ Kurtosis indicates the size of a distribution's tail. The higher the Kurtosis, the more likely it is that the outlier MI values differ substantially from values around the mean (Kurtosis>5 is considered significant for a sample size n<50)². K-S analysis was used to test coherence of the data-set to the normal distribution assumption.^{3,4} Significance indicates that the tested data-set is not normally distributed and values higher than the “most extreme positive difference” do significantly differ from the mean. Table 1 shows the distribution parameters of the measured methylation indices for each MINT locus and for each tissue category. MINT1, 12, and 25 methylation data distribution demonstrates high Kurtosis in adenomatous tissue while Kurtosis is lower than five in normal epithelium. K-S test becomes significant during adenomatous transformation for MINT1, 12, 17 and 25.¹⁻⁴

TABLE 1
Distribution results* of methylation index (MI) values in
normal, adenomatous and rectal cancer tissue

| | | | Tissue | | |
|---|---|---|---|---|---|
| | | | Normal | Adenoma | Cancer |
| Marker | | | n = 21 | n = 45 | n = 37 |
| MINT1 | Kurtosis | | 1.6 | 31.4 | 34.8 |
| | K-S analysis | Mean | 0.01 | 0.02 | 0.01 |
| | | Most extreme | 0.21 | 0.45 | 0.35 |
| | | positive | | | |
| | | difference | | | |
| | | P-Value | 0.31 | <0.001 | <0.001 |
| MINT2 | Kurtosis | | 20.9 | 3.9 | 3.4 |
| | K-S analysis | Mean | 0.01 | 0.10 | 0.05 |
| | | Most extreme | 0.49 | 0.35 | 0.38 |
| | | positive | | | |
| | | difference | | | |
| | | P-Value | <0.001 | <0.001 | <0.001 |
| MINT3 | Kurtosis | | 1.3 | 1.4 | 1.2 |
| | K-S analysis | Mean | 0.04 | 0.49 | 0.53 |
| | | Most extreme | 0.31 | 0.14 | 0.09 |
| | | positive | | | |
| | | difference | | | |
| | | P-Value | 0.03 | 0.36 | 0.92 |
| MINT12 | Kurtosis | | 0.7 | 21.8 | 25.7 |
| | K-S analysis | Mean | 0.03 | 0.04 | 0.04 |
| | | Most extreme | 0.21 | 0.33 | 0.35 |
| | | positive | | | |
| | | difference | | | |
| | | P-Value | 0.26 | <0.001 | <0.001 |
| MINT17 | Kurtosis | | 1.7 | 0.5 | 0.6 |
| | K-S analysis | Mean | 0.25 | 0.24 | 0.25 |
| | | Most extreme | 0.26 | 0.24 | 0.19 |
| | | positive | | | |
| | | difference | | | |
| | | P-Value | 0.12 | 0.01 | 0.04 |
| MINT25 | Kurtosis | | n.a. | 20.4 | 36.9 |
| | K-S analysis | Mean | 0.00 | 0.01 | 0.01 |
| | | Most extreme | 0.00 | 0.48 | 0.50 |
| | | positive | | | |
| | | difference | | | |
| | | P-Value | n.a. | <0.001 | <0.001 |
| MINT31 | Kurtosis | | 19.6 | 14.1 | 12.6 |
| | K-S analysis | Mean | 0.00 | 0.02 | 0.00 |
| | | Most extreme | 0.53 | 0.45 | 0.40 |
| | | positive | | | |
| | | difference | | | |
| | | P-Value | <0.001 | <0.001 | 0.002 |

*Kurtosis, Kolmogorov-Smirnov (K-S) Analysis

### REFERENCES

1. de Maat M F, Umetani N, Sunami E, et al: Assessment of methylation events during colorectal tumor progression by absolute quantitative analysis of methylated alleles. Mol Cancer Res 5:461-71, 2007

2. Lacher D A: Sampling distribution of skewness and kurtosis. Clin Chem 35:330-1, 1989

3. Young I T: Proof without prejudice: use of the Kolmogorov-Smirnov test for the analysis of histograms from flow systems and other sources. J Histochem Cytochem 25:935-41, 1977

4. Watson J V: Proof without prejudice revisited: immunofluorescence histogram analysis using cumulative frequency subtraction plus ratio analysis of means. Cytometry 43:55-68, 2001

### Supplemental Appendix 3

Comparison of Patient Characteristics
of the TME Trial and Selected Cases

| | | | | |
|---|---|---|---|---|
| | | Cases eligible | Analyzed | Non- |
| | Cases in | for current | cases for | analyzed |
| Character- | TME trial | study | clinical study | cases |
| istic | (n = 1805) | (n = 672) | (n = 251) | (n = 421) |

Age Category*

| | | | | |
|---|---|---|---|---|
| Median | 65 | 64 | 63 | 64 |

| | | | | |
|---|---|---|---|---|
| Range | 23-92 | 23-92 | 27-85 | 23-92 |

Sex*

| | | | | | | | | |
|---|---|---|---|---|---|---|---|---|
| Male | 1151 | (64) | 419 | (62) | 162 | (65) | 257 | (61) |
| Female | 654 | (36) | 253 | (38) | 89 | (35) | 164 | (39) |

Type of Resection*

| None | 45 | (2) | — | | — | | — | |
|---|---|---|---|---|---|---|---|---|
| Low anterior | 1183 | (66) | 442 | (66) | 167 | (66) | 275 | (65) |
| Abdomino- | 485 | (27) | 206 | (31) | 77 | (31) | 129 | (31) |
| perineal | | | | | | | | |
| Hartmann | 90 | (5) | 24 | (3) | 7 | (3) | 17 | (4) |

| | | | | | |
|---|---|---|---|---|---|
| Unknown | 2 | (<1) | — | — | — |

TNM stage*

| 0 | 28 | (2) | 11 | (2) | — | | 11 | (3) |
|---|---|---|---|---|---|---|---|---|
| I | 509 | (28) | 203 | (30) | 70 | (28) | 133 | (32) |
| II | 497 | (28) | 186 | (28) | 75 | (30) | 111 | (26) |
| III | 624 | (35) | 272 | (40) | 106 | (42) | 166 | (39) |

| | | | | | |
|---|---|---|---|---|---|
| IV | 122 | (7) | — | — | — |
| Unknown or | 25 | (1) | — | — | — |
| no resection | | | | | |

Circumferential Margin*

| | | | | | | | | |
|---|---|---|---|---|---|---|---|---|
| Negative | 1494 | (83) | 552 | (82) | 207 | (82) | 345 | (82) |
| Positive | 301 | (17) | 120 | (18) | 44 | (18) | 76 | (18) |

| | | | | | |
|---|---|---|---|---|---|
| Unknown | 10 | (<1) | — | — | — |

*P-value not significant; patients in current study were not significantly different from the original trial population.

### Example II

### Identification of a Quantitative MINT Locus Methylation Profile Predicting Local Recurrence of Rectal Cancer

### Abstract

Purpose: In preoperative treatment planning for patients undergoing primary tumor resection for rectal cancer, the major clinical problem is who will be at high risk for loco-regional disease recurrence. Epigenetic aberrations such as DNA methylation have been shown to be significant prognostic biomarkers of disease outcome. In this study the significance of a quantitative epigenetic multi-marker panel analysis of primary tumors to predict local recurrence in rectal cancer patients from a multicenter phase III clinical trial was evaluated. Methods: Methylation levels of seven methylated-in-tumor (MINT) loci were assessed by absolute quantitative assessment of methylated alleles (AQAMA) in primary tumors from patients that underwent total mesorectal excision (TME) for primary rectal cancer (n=251). Unsupervised random forest clustering of quantitative MINT methylation data was used to show subclassification into groups with matching methylation profiles. Results: Variable importance parameters (Gini-Index) of the clustering algorithm indicated MINT 3 and 17 (GI=20.2 and 20.7, respectively) to be most informative for patient grouping compared to the other MINT loci (highest GI 12.2). When using this two-marker panel, four different patient clusters could be identified. One patient group (73%) was at significantly increased risk of local recurrence (hazard ratio 10.23, 95% confidence interval 1.38-75.91) in multivariate analysis, corrected for standard prognostic factors of rectal cancer. This group had a significant higher local recurrence probability than patients receiving preoperative radiation (P<0.0001). Conclusion: Quantitative epigenetic subclassification of rectal cancers has clinical utility in distinguishing tumors with increased risk for local recurrence and may help tailor treatment regimens for locoregional control.

### Introduction

Rectal cancer is the second most common cancer of the digestive system in the USA¹. Worldwide, colorectal cancer incidence takes a second place², with rectal tumors constituting 33% of large bowel tumors³. An important clinical feature of rectal cancer is its close anatomical relation to the small pelvis which makes it prone to local recurrence after surgical removal even at early stage disease. Local recurrence occurs in approximately 10% of rectal cancer patients after total mesorectal excision surgery with curative intent^{4,5}.

Fixation in the small pelvis makes malignancies of the rectum suitable for external beam radiation therapy. The multidisciplinary treatment of rectal cancer is a subject of many clinical trials randomizing patients to regimens that include various (neo) adjuvant therapies combined with radical surgery⁶⁻⁹. The Dutch multicenter total mesorectal excision (TME) clinical trial demonstrated significant reduction of local recurrence by adding short course (5×5Gy) preoperative radiation therapy to TME¹⁰.

(Neo)adjuvant regimens aim at two clinical outcome parameters: improvement of local control and/or to reduce distant recurrence, the latter occurring in approximately 25% of patients after radical primary tumor resection⁴. The improved local control shown by the TME clinical trial has not translated into an overall survival benefit in the trial analyses after 6 years of follow-up and the data show that overall survival is determined predominantly by distant recurrence⁴. The trial data further show that only 25% of non-irradiated tumors with distant spread also recur locally, whereas 60% of the locally recurrent tumors show distant disease spread^{4,10}. The distant-spreading, non-locally recurring tumor may therefore constitute a separate subclass of rectal cancer.

Using adjuvant chemotherapy, a reduction of distant recurrence of rectal cancer, as well as improved disease-free and overall survival, have been shown in several randomized controlled trials, either alone or in combination with radiotherapy¹¹⁻¹³. Allocation of patients to (neo)adjuvant therapies might lead to overtreatment; since 10% of rectal cancer patients will develop local recurrence, 90% of patients may be overtreated. Neoadjuvant therapies used in the treatment of rectal cancer have their specific morbidities, as has been shown for both radiotherapy¹⁴ and combined chemoradiotherapy¹⁵. It is therefore of great importance to define biomarkers that can categorize tumors into local and distant spreading type preoperatively to target the multimodality treatment regimens towards a more systemic or local approach.

One of the most important diagnostic parameters to date is lymph node status, but this can only be reliably assessed post-operatively¹⁶. Measurements of serum carcinoembryonic antigen (CEA) levels were also shown to correlate with disease recurrence after curative resection for rectal cancer¹⁷. However, at least 50% of patients with a normal pre-operative CEA levels show recurrent disease¹⁷. Molecular analysis of primary tumor tissues is an attractive form of preoperative diagnostics since rectal primary tumors are easily accessible for biopsy, which is routinely performed in the preoperative work-up. Molecular biomarkers suggested to have prognostic value in colorectal cancer include loss of heterozygosity at chromosome 18q¹⁸, microsatellite instability¹⁹ and K-ras²⁰, among others. In contrast to colorectal cancer, to date few biomarkers have been described that are predictive or prognostic value specifically in rectal cancer. To distinguish rectal cancer from other large bowel adenocarcinomas is important especially in multivariate analyses. Specific factors need to be taken into account for rectal cancer as compared to colon cancer, such as circumferential margin, type of surgical procedure or distance of the tumor to the anal verge²¹.

Epigenetic changes, such as changes in DNA methylation status, are regarded as early events contributing to carcinogenesis²². Methylation of cytosine residues in DNA is one of the mechanisms regulating transcriptional activity²³. In cancer, aberrant DNA hypermethylation of specific regions as well as global hypomethylation is observed²⁴. In this study, we investigated epigenetic changes in rectal cancers and specifically CpG methylation of methylated-in-tumor (MINT) loci. MINT loci are CpG dinucleotide-rich regions located in nonprotein-encoding DNA regions and have been reported to become methylated in a tumor-specific manner in (colo)rectal cancer^{25,26}, gastric cancer²⁷ and recently malignant melanoma²⁸.

In a previous study, we have quantitatively studied methylation of MINT loci in premalignant stages of rectal cancer and have shown that the MINT methylation index increases during adenomatous transformation of normal epithelium²⁶. We also demonstrated that in primary rectal cancer tissues of TME clinical trial patients, methylation levels of a multi-marker methylation panel are predictive of distant recurrence in early, node-negative rectal cancers²⁶. In the present study, we assessed the value of the multi-marker methylation panel to predict local recurrence using paraffin-embedded archival tissue (PEAT) sections from 251 patients enrolled in the multicenter, randomized, quality-controlled clinical TME trial. Using the quantitative methylation data from AQAMA, unsupervised cluster analysis identified a subgroup of patients at increased risk for local recurrence when node negative based on MINT locus methylation-level differences.

### Methods

### Tissue Specimens

The study population was established by sample size calculations (see Results section). Primary PEAT specimens were obtained from 325 non-irradiated patients enrolled in the TME clinical phase-III trial⁴. Patients used in this study fulfilled the following criteria: non-irradiated, TNM stage I-III, with no evidence of disease after surgical resection. The selected group of patients analyzed in our previous study²⁶ did not to differ from non-selected patients in the non-irradiated treatment arm or from the complete trial population. Trial eligibility criteria and follow-up protocols have been described previously^{4,29,30}. Non-irradiated patients were selected since predictive value of the tested biomarkers for local recurrence probability should be tested in patients who did not receive adjuvant therapy, as this affects local recurrence.

DNA Preparation from PEAT Specimens

From the TME trial patient's primary tumor PEAT specimens, 7-μm tissue sections were cut and mounted on non-adhesive glass slides. Tumor-representative areas on H&E-stained sections were identified and marked by a surgical pathologist specializing in rectal cancer (JHJMvK). Per patient, two tissues sections were deparaffinized. DNA was isolated from microdissected tissue from the marked areas and modified by sodium bisulfite, as previously described³¹. Salmon sperm DNA was added as a carrier³². Before and after sodium bisulfite modification, double-stranded and single-stranded DNA were quantified using PicoGreen and OliGreen assays (Molecular Probes; Invitrogen, Carlsbad, Calif.), respectively. Sufficient input DNA for AQAMA was determined as described²⁵. To assess background signal, a salmon sperm DNA sample without tumor DNA was included in all assays in triplicate. To prevent any bias, tissue blocks and isolated DNA were coded.

### AQAMA MINT Locus Methylation Level Assessment

In 251 TME trial patients, primary tumor methylation levels of methylated-in-tumor (MINT) loci 1, 2, 3, 12, 17, 25 and 31 were assessed by AQAMA in triplicate²⁵. Controls for specificity of AQAMA for both methylated and unmethylated sequences, as well as controls for nonspecific amplification, were included^{25,33}. As a final outcome of the analysis, a sample's methylation index (MI) was calculated as follows: [copy number_{methylated alleles}/(copy number_{methylated alleles}+copy number_{unmethylated alleles})].

### Profiling by Unsupervised Random Forest Clustering

Unsupervised random forest (RF) clustering and an expectation-maximization mixture of Gaussians (EM-MoG) algorithm subclassified the patients into groups with matching methylation profiles, as described previously^{26,34}. The random forest algorithm is a data-driven approach based on individual decision-tree predictors that automatically dichotomizes expression levels into clusters. Groups were therefore not established using cutoff values. Cluster assignment was obtained using EM-MoG analysis, which is based on a two-step approach to fit Gaussian probability models on the data. Most likely clusters were represented in a multidimensional scaling (MDS) plot. Correction for probability of cluster allocation was performed by multiple imputation analysis³⁵. Local recurrence probabilities were evaluated for the identified patient groups.

### Statistical Analysis

Differences in recurrence probability, survival and clinical and tumor pathologic factors were analyzed between TME trial patients assigned by RF clustering. Specimens yielding insufficient or low quality DNA for polymerase chain reaction were excluded. To compare ordinal variables, Mann-Whitney and Kruskal-Wallis U tests were performed. Differences in age were assessed using t tests. The Kaplan-Meier method was used to visualize survival differences and significance was assessed by the log-rank test. For multivariate analysis the Cox proportional hazards model was used, with results presented as hazard ratios and 95% confidence intervals. Co-variables entered in the model included T stage, N stage, circumferential margin status, distance of the tumor from the anal verge and tumor differentiation. All clinical correlative analyses with identified clusters were performed using SPSS statistical software (version 16.0.1, SPSS Inc, Chicago, Ill.). A two-sided p-value of 0.05 or less was considered statistically significant. Data on patients alive or free of recurrence were censored at the time of the last follow-up.

### Results

### Patient Specimens Analyzed

Using power calculations, a sample size of 250 patients was calculated to be sufficient to obtain statistical significance for predicting recurrence (with α=0.05 and a power of 90%), as described previously²⁶. Allowing for 30% loss of patient samples due to availability and quality of paraffin tissue and DNA, 75 additional tissue samples were collected. In eleven patient tissue blocks, tumor tissue was no longer present on the section. Finally, DNA was isolated from 314 randomly selected patient samples with sufficient tumor cell numbers. After processing and sodium bisulfite treatment, samples of 251 patients yielded sufficient input DNA for AQAMA.

### MINT Locus Methylation Profiling

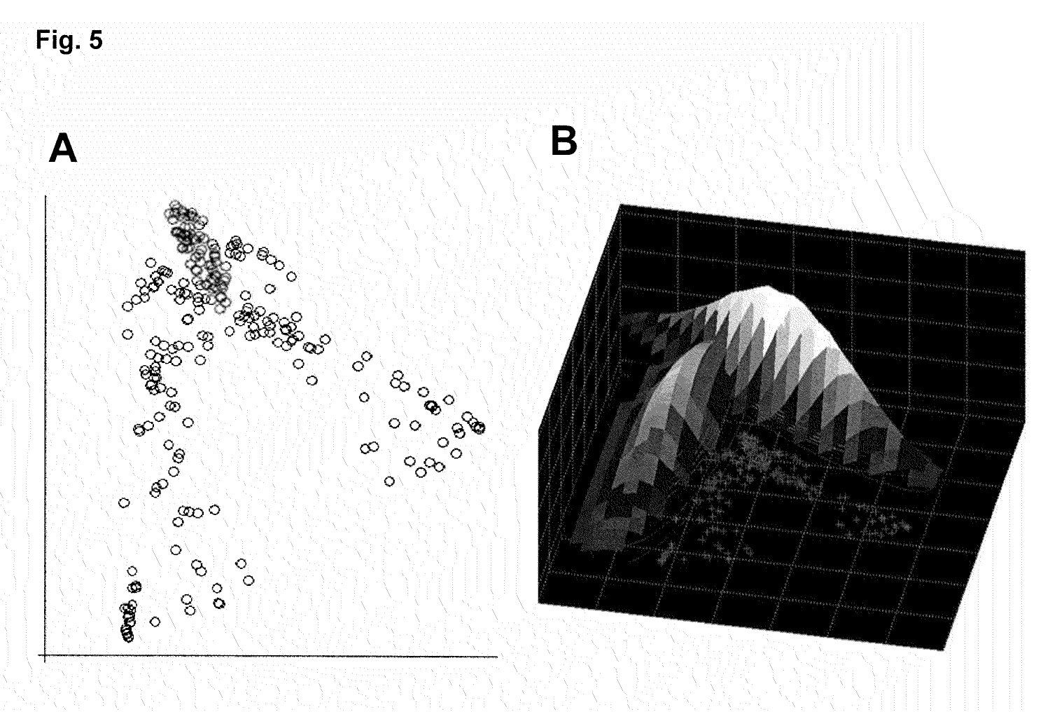

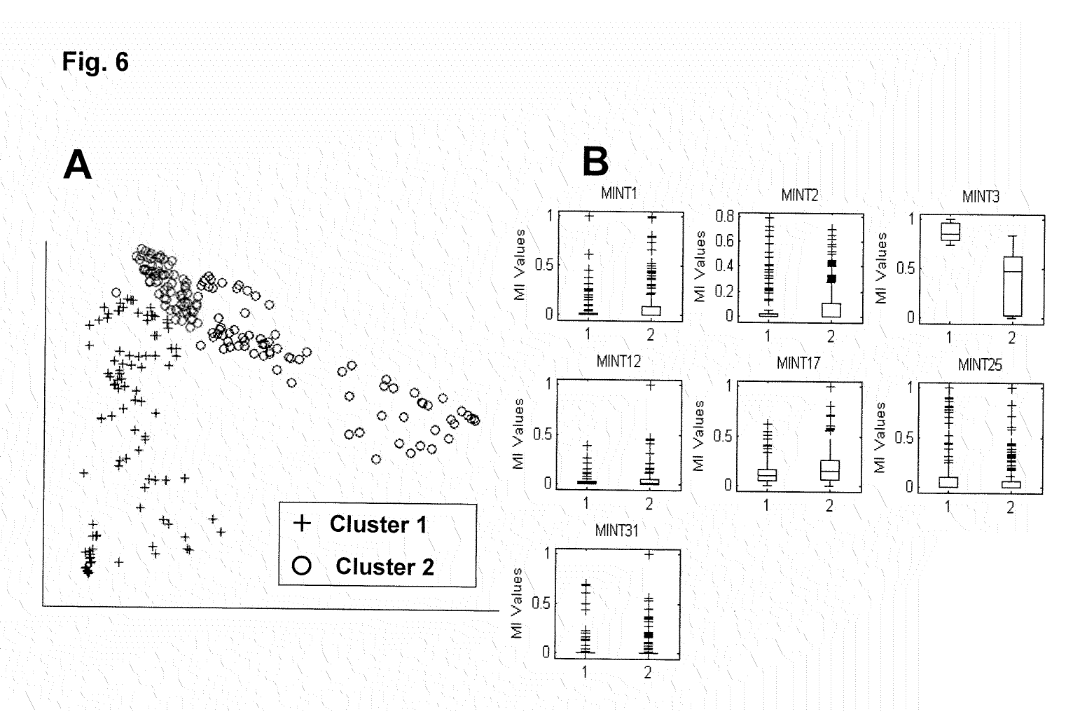

In a previous study methylation levels at the seven MINT loci were measured in normal, adenomatous and cancer rectal tissue²⁶. The results showed that in normal tissue, all MINT loci, except for MINT17, were mostly unmethylated. Significantly higher methylation of MINT2, 3 and 31 was detected in adenoma and cancer tissue compared to normal tissue. Further analysis of the quantitative data showed non-parametric distributions indicating presence of subgroups for MINT1, 2, 12, 17, 25 and 31 in adenoma and cancer tissue. Based on these findings we concluded that all seven MINT loci had potential to subclassify patient groups with corresponding methylation level patterns. Quantitative methylation data of the seven MINT loci were then used to perform unsupervised RF clustering analysis, with a two dimensional MDS plot as an outcome. The mutual distance between the dots, representing individual patients, indicates MINT methylation level correspondence (FIG. 5). As a second step an EM-MoG algorithm assigned the patients to two clusters that were present within the data based on quantitative methylation levels (FIG. 6). One patient group (cluster 2) showed significantly decreased methylation at MINT3 (P<0.001) and significantly increased methylation at MINT1, 12 and 17 (P<0.001, P=0.01 and P<0.001, respectively) compared with patients in cluster 1 (FIG. 6B).

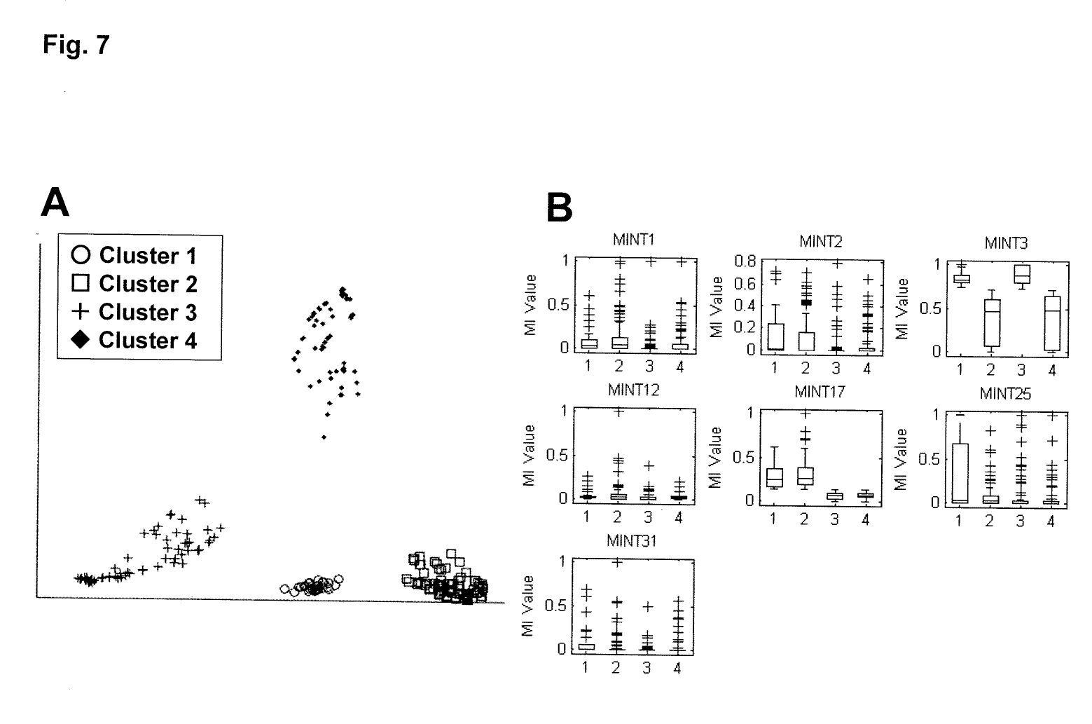

The Gini-index of the RF-analysis, indicating variable importance, appointed MINT3 and MINT17 as the two MINT markers that carried the most information to form the clusters²⁶. Being a measure of inequality, the higher the Gini-index, the more the clusters can be considered different based on that specific biomarker. MINT3 and MINT17 were shown to have the highest Gini-index (20.2 and 20.7, respectively), compared to the other MINT loci (range 6.0-13.5)²⁶. The multidimensional scaling (MDS) plot of the RF clustering using only these two markers showed four separate groups (FIG. 7). Patients could now be definitely allocated to cluster because of the clear separation of the patient groups in the plot, without further correction of results for cluster uncertainty. The patient group that corresponded to the methylation pattern using all seven MINT loci with significantly increased methylation at MINT3 and significantly decreased methylation at MINT17 (cluster 2; see ²⁶), was identified (cluster 3) and consisted of 68 patients (27%). The patient group showing decreased methylation at MINT3 and increased methylation at MINT17 was identified, corresponding with cluster 2.

### Univariate Analysis of Clinicopathological Parameters

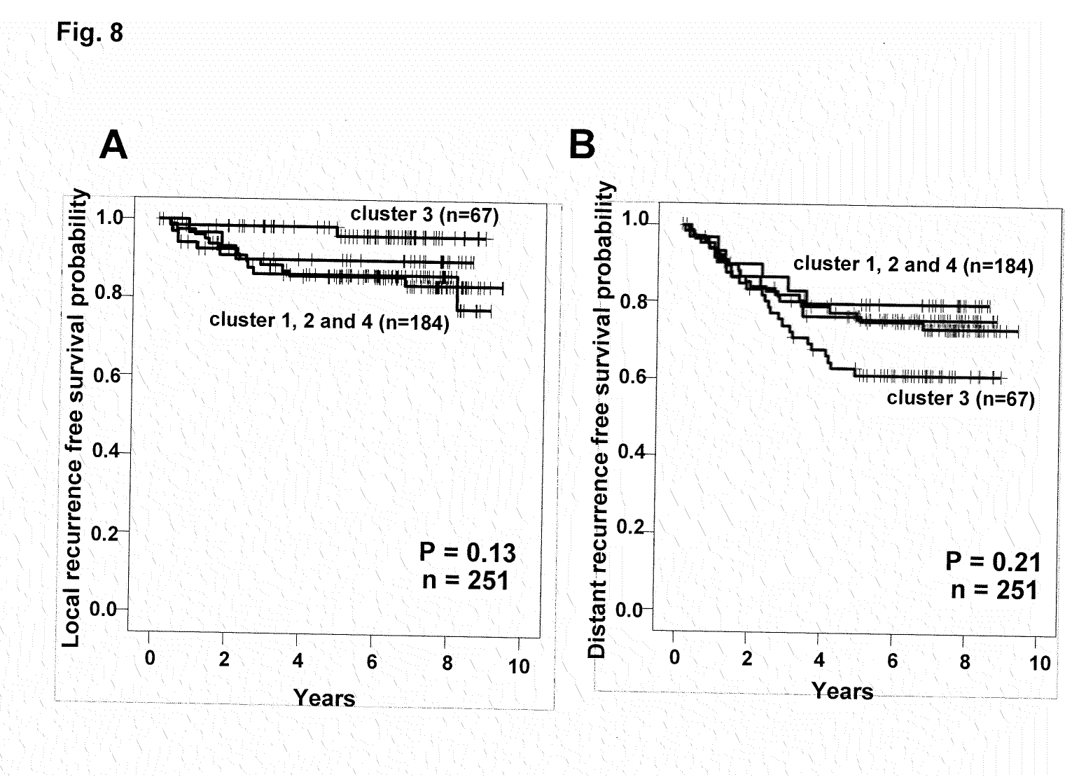

Next, we were interested in comparing probability of local recurrence between the four clusters. As shown in FIG. 8A, local recurrence free probability was highest for patients in cluster 3 having the specific combination of high MINT3 and low MINT17 methylation compared to the other clusters (P=0.13). Clusters 1, 2 and 4 showed similar probability outcomes. The difference in local recurrence probability became more evident and reached statistical significance after combining clusters 1, 2 and 4 (P=0.03; FIG. 8B). This result shows that the specific combination of increased methylation at MINT3 and decreased methylation at MINT17 is predictive of reduced local recurrence probability.

Using univariate analysis, standard clinicopathological parameters including sex, age, TNM stage, N-status, tumor differentiation, location of distant recurrences, resection type and circumferential margin status were compared between cluster 3 and the combined group of clusters 1, 2 and 4. These parameters did not significantly differ between the two patient groups identified based on methylation levels of MINT3 and MINT17 (Table 1). In addition, no significant difference was observed for these standard clinicopathological factors between the patient groups when nodal status was taken into account (Table 2).

TABLE 1
Comparison of clinical and tumor pathology factors between
identified two MINT locus clusters in all patients

| M3 & M17 | | |
|---|---|---|
| Cluster | Cluster | |
| 1, 2 and 4 | 3 | |
| n = 184 | N = 67 | P-Value |

Sex

| | | | | | |
|---|---|---|---|---|---|
| Male | 116 | (72) | 46 | (28) | 0.41 |
| Female | 68 | (76) | 21 | (24) | |

Age

| | | | | | |
|---|---|---|---|---|---|
| Mean (SE) | 62.7 | (0.9) | 65.0 | (1.4) | 0.17 |

TNM-Stage

| | | | | | |
|---|---|---|---|---|---|
| I | 51 | (73) | 19 | (27) | 0.78 |
| II | 57 | (76) | 18 | (24) | |
| III | 76 | (72) | 30 | (28) | |

N-Status

| | | | | | |
|---|---|---|---|---|---|
| N₀ (≧12 examined) | 27 | (79) | 7 | (21) | 0.67 |
| N₀/N_{X} (<12 examined) | 82 | (73) | 30 | (27) | |
| N₁ (1-3 positive) | 44 | (72) | 17 | (28) | |
| N₂ (≧4 positive) | 32 | (73) | 12 | (27) | |

Differentiation

| | | | | | |
|---|---|---|---|---|---|
| Well | 14 | (78) | 4 | (22) | 0.90 |
| Moderately | 128 | (73) | 48 | (27) | |
| Poor | 42 | (74) | 15 | (26) | |

Location Distant Recurrence

| | | | | | |
|---|---|---|---|---|---|
| Liver | 19 | (70) | 8 | (30) | 0.44 |
| Non-liver | 24 | (59) | 17 | (41) | |

Resection Type

| | | | | | |
|---|---|---|---|---|---|
| Low anterior | 125 | (75) | 42 | (25) | 0.46 |
| Abdominoperineal | 53 | (69) | 24 | (31) | |
| Hartmann | 6 | (86) | 1 | (14) | |

Circumferential Margin

| | | | | | |
|---|---|---|---|---|---|
| Negative | 152 | (73) | 55 | (27) | 1 |
| Positive | 32 | (73) | 12 | (27) | |

TABLE 2
Comparison of clinical and tumor pathology factors
between two MINT loci clusters in node-negative and node-positive
patients

| | M3 & M17 | | | M3 & M17 | | |
|---|---|---|---|---|---|---|
| | Node Negative | | | Node Positive | | |
| | (n = 145) | | | (n = 106) | | |
| | Cluster 1, | | | Cluster 1, | | |
| | 2 & 4 | Cluster 3 | | 2 & 4 | Cluster 3 | |
| Clinical Parameters | n = 108 | n = 37 | P | n = 76 | n = 30 | P |
| Sex | | | | | | |
| Male | 116 (72) | 46 (28) | 0.41 | 116 (72) | 46 (28) | 0.41 |
| Female | 68 (76) | 21 (24) | | 68 (76) | 21 (24) | |
| Age | | | | | | |
| Mean (SE) | 62.7 (0.9) | 65.0 (1.4) | 0.17 | 62.7 (0.9) | 65.0 (1.4) | 0.17 |
| TNM-Stage | | | | | | |
| I | 51 (73) | 19 (27) | 0.78 | 51 (73) | 19 (27) | 0.78 |
| II | 57 (76) | 18 (24) | | 57 (76) | 18 (24) | |
| III | 76 (72) | 30 (28) | | 76 (72) | 30 (28) | |
| N-Status | | | | | | |
| N₀ (≧12 examined) | 27 (79) | 7 (21) | 0.67 | 27 (79) | 7 (21) | 0.67 |
| N₀/Nₓ (<12 examined) | 82 (73) | 30 (27) | | 82 (73) | 30 (27) | |
| N₁ (1-3 positive) | 44 (72) | 17 (28) | | 44 (72) | 17 (28) | |
| N₂ (≧4 positive) | 32 (73) | 12 (27) | | 32 (73) | 12 (27) | |
| Differentiation | | | | | | |
| Well | 14 (78) | 4 (22) | 0.90 | 14 (78) | 4 (22) | 0.90 |
| Moderately | 128 (73) | 48 (27) | | 128 (73) | 48 (27) | |
| Poor | 42 (74) | 15 (26) | | 42 (74) | 15 (26) | |
| Location Distant | | | | | | |
| Recurrences | | | | | | |
| Liver | 19 (70) | 8 (30) | 0.44 | 19 (70) | 8 (30) | 0.44 |
| Non-liver | 24 (59) | 17 (41) | | 24 (59) | 17 (41) | |
| Resection Type | | | | | | |
| Low anterior | 125 (75) | 42 (25) | 0.46 | 125 (75) | 42 (25) | 0.46 |
| Abdominoperineal | 53 (69) | 24 (31) | | 53 (69) | 24 (31) | |
| Hartmann | 6 (86) | 1 (14) | | 6 (86) | 1 (14) | |
| Circumferential Margin | | | | | | |
| Negative | 152 (73) | 55 (27) | 1 | 152 (73) | 55 (27) | 1 |
| Positive | 32 (73) | 12 (27) | | 32 (73) | 12 (27) | |

### Multivariate Analyses

To assess whether the observed prognostic value of the clusters was independent from standard prognostic variables, we performed multivariate analyses. The Cox regression method was used to analyze standard prognostic factors of rectal cancer; T stage, N stage, circumferential margin status, distance of the tumor from the anal verge and tumor differentiation (Table 3).

TABLE 3
Multivariate analyses in all analyzed patients

| | All Patients | | | | | |
|---|---|---|---|---|---|---|
| | HR (95% CI) | P | HR (95% CI) | P | HR (95% CI) | P |
| | Local | | Cancer Specific | | Overall | |
| Variable | Recurrence | | Survival | | Survival | |
| T-Stage (3-4) | 1.16 | 0.76 | 2.08 | 0.04 | 1.89 | 0.02 |
| | (0.44-3.08) | | (1.05-4.12) | | (1.13-3.18) | |
| Node (+) | 3.40 | 0.007 | 2.43 | 0.002 | 1.86 | 0.005 |
| | (1.39-8.33) | | (1.38-4.29) | | (1.20-2.89) | |
| Circumferential | 2.27 | 0.07 | 1.90 | 0.03 | 1.62 | 0.05 |
| Margin (+) | (0.93-5.53) | | (1.08-3.35) | | (1.00-2.64) | |
| Distance from Anal | 1.40 | 0.42 | 1.71 | 0.04 | 1.47 | 0.08 |
| Verge <5 cm | (0.62-3.18) | | (1.01-2.88) | | (0.96-2.24) | |
| Poor Differentiation | 0.98 | 0.96 | 1.54 | 0.12 | 1.31 | 0.25 |
| | (0.40-2.41) | | (0.90-2.65) | | (0.83-2.06) | |
| MINT Locus | 10.23 | 0.02 | 1.43 | 0.18 | 1.41 | 0.12 |
| Profile* | (1.38-75.91) | | (0.84-2.44) | | (0.91-2.17) | |

HR: hazard ratio,
CI: confidence interval.
*“Cluster 1, 2 or 4” is null-hypothesis.

Based on the epigenetic subclassification, the multivariate analysis showed patients of clusters 1, 2 or 4 to be at significant, over 10-fold increased risk on local recurrence (Table 3). Nodal stage was significantly associated with local recurrence, cancer specific survival and overall survival. T stage and circumferential margin status significantly affected cancer-specific and overall survival. Distance from the anal verge (<5 cm) only affected cancer-specific survival. When subdivided according to nodal status, patients in cluster 3 with a positive nodal status were at significantly increased risk of local recurrence (Table 4). Node negative patients in cluster 3 showed a significantly decreased cancer-specific and overall survival compared to the other clusters.

TABLE 4
Multivariate analyses for patients subdivided
into node-negative and node-positive

| Node Negative | | Node Positive | |
|---|---|---|---|
| (n = 145) | | (n = 106) | |
| HR (95% CI) | P | HR (95% CI) | P |

Local Recurrence

| | | | | | | |
|---|---|---|---|---|---|---|
| T-Stage | 0.48 | (0.09-2.77) | 0.42 | 1.98 | (0.44-8.96) | 0.38 |
| Circumferential | 6.42 | (0.89-46.03) | 0.07 | 1.99 | (0.75-5.29) | 0.17 |
| Margin (+) | | | | | | |
| Distance from | 3.73 | (0.45-30.86) | 0.22 | 2.63 | (1.06-6.55) | 0.04 |
| Anal | | | | | | |
| Verge <5 cm | | | | | | |
| Poor Differ- | 0.83 | (0.10-7.14) | 0.86 | 1.08 | (0.39-2.96) | 0.88 |
| entiation | | | | | | |

| | | | | | |
|---|---|---|---|---|---|
| MINT Locus | n.a. | 0.97 | 7.68 | (1.02-57.78) | 0.05 |
| Profile† | | | | | |

Cancer Specific Survival

| | | | | | | |
|---|---|---|---|---|---|---|
| T-Stage | 1.81 | (0.68-4.82) | 0.24 | 2.90 | (1.00-8.40) | 0.05 |
| Circumferential | 2.09 | (0.55-7.99) | 0.28 | 1.96 | (1.04-3.68) | 0.04 |
| Margin (+) | | | | | | |
| Distance from | 1.39 | (0.51-3.77) | 0.52 | 2.58 | (1.40-4.75) | 0.002 |
| Anal | | | | | | |
| Verge <5 cm | | | | | | |
| Poor Differ- | 1.32 | (0.41-4.25) | 0.64 | 1.65 | (0.86-3.16) | 0.13 |
| entiation | | | | | | |
| MINT Locus | 0.30 | (0.12-0.75) | 0.009 | 0.92 | (0.47-1.78) | 0.80 |
| Profile† | | | | | | |

Overall Survival

| | | | | | | |
|---|---|---|---|---|---|---|
| T-Stage | 2.04 | (1.01-4.11) | 0.05 | 2.01 | (0.88-4.56) | 0.10 |
| Circumferential | 1.55 | (0.57-4.21) | 0.40 | 1.72 | (0.99-2.99) | 0.06 |
| Margin (+) | | | | | | |
| Distance from | 0.94 | (0.48-1.83) | 0.85 | 1.88 | (1.10-3.24) | 0.02 |
| Anal | | | | | | |
| Verge <5 cm | | | | | | |
| Poor Differ- | 1.07 | (0.46-2.50) | 0.88 | 1.35 | (0.77-2.38) | 0.30 |
| entiation | | | | | | |
| MINT Locus | 0.46 | (0.24-0.89) | 0.02 | 0.87 | (0.49-1.53) | 0.62 |
| Profile† | | | | | | |

†:“Cluster 1, 2 and 4” is null hypothesis.
Ns: not significant, n.a.: not assessable, HR: hazard ratio, CI: confidence interval.

Comparison with Preoperatively Irradiated Tumors

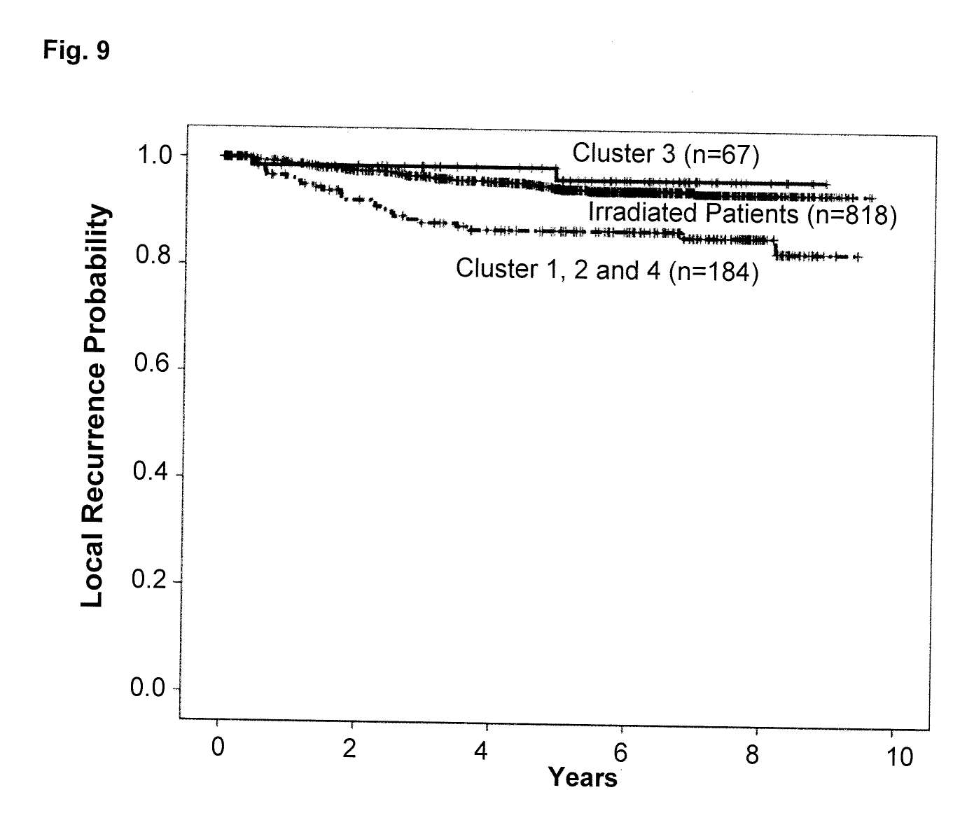

Because we found the non-irradiated cluster 3 patients to be at reduced risk for local recurrence we were interested to see how recurrence probability rates of this group compared to those of preoperatively irradiated patients from the TME trial (FIG. 9). The irradiated patients were selected using the same clinical parameters that did not differ significantly from the non-irradiated selected patients. The local recurrence rate in cluster 3 patients was 3% versus 4.8% in patients who receive 5×5 Gy before surgery (P=0.43). Patients of high-risk clusters 1, 2 or 4 had significantly higher recurrence probability over time postoperatively compared to irradiated patients (P<0.0001). This result indicates that patients without preoperative radiation therapy in patients identified by the quantitative 2 marker MINT profile have at least comparable, maybe improved local recurrence rates compared to irradiated patients and will likely not be disadvantaged when preoperative radiotherapy is left out.

### Discussion

Although recurrence rates have decreased to 10% after the introduction of TME surgery, locally recurrent cancer remains an important clinical problem. Preoperative molecular profiling of the primary tumor could potentially be of great value to identify patients at high risk of developing local recurrence. This study shows that based on absolute quantitative methylation levels of the MINT3 and MINT17 loci, rectal cancers with a high-risk of local recurrence can be identified.

Based on power calculations, methylation levels of seven MINT loci were determined in 251 patient samples using AQAMA. In a previous study²⁶, MINT methylation was shown to increase early during tumor progression, indicating that methylation of MINT loci is a factor acquired early during rectal tumorigenesis, and therefore can be used to subclassify early disease. The subsequent grouping of patients based on MINT methylation levels was demonstrated to be clinically relevant, as MINT loci (especially MINT3 and MINT17) were shown to have prognostic value in predicting progression to distant disease²⁶. In this study we used only MINT3 and MINT17 as these were most informative in RF clustering, which resulted in four separate patient clusters. One of the patient groups (cluster 3) showed a methylation pattern corresponding to the previously described pattern for cluster 1 (increased MINT3 and decreased MINT17 methylation)²⁶. We also showed that local recurrence rates of cluster 3 patients are comparable to irradiated rectal cancer patients and this shows that leaving out preoperative radiation can be done safely with the advantage of reducing treatment morbidity.

Non-irradiated tumors that show local recurrence or spread distantly show different effects on clinical outcomes. The patients of cluster 3 show a significantly increased risk of distant recurrence²⁶ and significantly reduced risk on local recurrence. This suggests that non-locally recurrent and distantly spreading rectal cancer constitutes a separate subclass of rectal cancers which can be identified by our two-marker MINT methylation profile.

Subdivision according to nodal status showed that patients in cluster 3 with a positive nodal status were at significant increased risk of local recurrence. Node negative patients in cluster 3, however, showed a significantly decreased cancer-specific and overall survival compared to the other clusters²⁶. This is explained by the fact that these node negative patients were found to be at significantly increased risk of distant recurrence in our previous study²⁶. These findings would be in accordance with data from the TME trial showing that survival is determined predominantly by distant and not by local recurrences. Early metastasizing of rectal cancer may occur via haematogenic spreading; this is supported by the fact that circulating tumor cells can be detected in peripheral blood of early stage I and II colorectal cancer (Koyanagi et al., 2008, Clin Cancer Res. 14(22):7391-6).

This is the first study to demonstrate that rectal cancer local recurrence patterns can be distinguished using quantitative epigenetic subclassification of primary rectal tumor tissue. In addition, this study shows that the methylation status of the described MINT loci can be used as a, preoperatively assessable, clinical biomarker with predictive value for rectal cancer specifically. Based on the results of this study, a new treatment stratification approach can be suggested as follows: after preoperative assessment of primary tumor MINT3 and MINT17 methylation levels about 30% of patients could be spared from preoperative radiation therapy, but might benefit from systemic treatment. The other 70% should receive preoperative radiotherapy, and if node-positive, postoperatively systemic treatment can be considered.

### REFERENCES

1. Jemal A, Siegel R, Ward E, et al: Cancer statistics, 2008. CA Cancer J Clin 58:71-96, 2008

2. Garcia M J A, Ward E M, et al.: Global cancer facts & figures 2007. Atlanta (Ga.): American Cancer Society 2007., 2007

3. Coleman M P, Quaresma M, Berrino F, et al: Cancer survival in five continents: a worldwide population-based study (CONCORD). Lancet Oncol 9:730-56, 2008

4. Peeters K C, Marijnen C A, Nagtegaal I D, et al: The TME trial after a median follow-up of 6 years: increased local control but no survival benefit in irradiated patients with resectable rectal carcinoma. Ann Surg 246:693-701, 2007

5. Das P, Skibber J M, Rodriguez-Bigas M A, et al: Clinical and pathologic predictors of locoregional recurrence, distant metastasis, and overall survival in patients treated with chemoradiation and mesorectal excision for rectal cancer. Am J Clin Oncol 29:219-24, 2006

6. Kapiteijn E, Marijnen C A, Nagtegaal I D, et al: Preoperative radiotherapy combined with total mesorectal excision for resectable rectal cancer. N Engl J Med 345:638-46, 2001

7. Cedermark B, Johansson H, Rutqvist L E, et al: The Stockholm I trial of preoperative short term radiotherapy in operable rectal carcinoma. A prospective randomized trial. Stockholm Colorectal Cancer Study Group. Cancer 75:2269-75, 1995

8. O'Connell M J: Combined-modality neoadjuvant therapy for rectal cancer. J Clin Oncol 23:5450-1, 2005

9. Bosset J F, Collette L, Calais G, et al: Chemotherapy with preoperative radiotherapy in rectal cancer. N Engl J Med 355:1114-23, 2006

10. Bakx R, Visser O, Josso J, et al: Management of recurrent rectal cancer: a population based study in greater Amsterdam. World J Gastroenterol 14:6018-23, 2008

11. Akasu T, Moriya Y, Ohashi Y, et al: Adjuvant chemotherapy with uracil-tegafur for pathological stage III rectal cancer after mesorectal excision with selective lateral pelvic lymphadenectomy: a multicenter randomized controlled trial. Jpn J Clin Oncol 36:237-44, 2006

12. Braendengen M, Tveit K M, Berglund A, et al: Randomized phase III study comparing preoperative radiotherapy with chemoradiotherapy in nonresectable rectal cancer. J Clin Oncol 26:3687-94, 2008

13. Taal B G, Van Tinteren H, Zoetmulder F A: Adjuvant 5FU plus levamisole in colonic or rectal cancer: improved survival in stage II and III. Br J Cancer 85:1437-43, 2001

14. Marijnen C A, van de Velde C J, Putter H, et al: Impact of short-term preoperative radiotherapy on health-related quality of life and sexual functioning in primary rectal cancer: report of a multicenter randomized trial. J Clin Oncol 23:1847-58, 2005

15. Buie W D, MacLean A R, Attard J A, et al: Neoadjuvant chemoradiation increases the risk of pelvic sepsis after radical excision of rectal cancer. Dis Colon Rectum 48:1868-74, 2005

16. Lahaye M J, Engelen S M, Nelemans P J, et al: Imaging for predicting the risk factors—the circumferential resection margin and nodal disease—of local recurrence in rectal cancer: a meta-analysis. Semin Ultrasound CT MR 26:259-68, 2005

17. Grossmann I, de Bock G H, Meershoek-Klein Kranenbarg W M, et al: Carcinoembryonic antigen (CEA) measurement during follow-up for rectal carcinoma is useful even if normal levels exist before surgery. A retrospective study of CEA values in the TME trial. Eur J Surg Oncol 33:183-7, 2007

18. Jen J, Kim H, Piantadosi S, et al: Allelic loss of chromosome 18q and prognosis in colorectal cancer. N Engl J Med 331:213-21, 1994

19. Lanza G, Gafa R, Santini A, et al: Immunohistochemical test for MLH1 and MSH2 expression predicts clinical outcome in stage II and III colorectal cancer patients. J Clin Oncol 24:2359-67, 2006

20. Andreyev H J, Norman A R, Cunningham D, et al: Kirsten ras mutations in patients with colorectal cancer: the ‘RASCAL II’ study. Br J Cancer 85:692-6, 2001

21. den Dulk M, Marijnen C A, Putter H, et al: Risk factors for adverse outcome in patients with rectal cancer treated with an abdominoperineal resection in the total mesorectal excision trial. Ann Surg 246:83-90, 2007

22. Counts J L, Goodman J I: Alterations in DNA methylation may play a variety of roles in carcinogenesis. Cell 83:13-5, 1995

23. Jones P A, Takai D: The role of DNA methylation in mammalian epigenetics. Science 293:1068-70, 2001

24. Das P M, Singal R: DNA methylation and cancer. J Clin Oncol 22:4632-42, 2004

25. de Maat M F, Umetani N, Sunami E, et al: Assessment of methylation events during colorectal tumor progression by absolute quantitative analysis of methylated alleles. Mol Cancer Res 5:461-71, 2007

26. de Maat M F, van de Velde C J, van der Werff M P, et al: Quantitative analysis of methylation of genomic loci in early-stage rectal cancer predicts distant recurrence. J Clin Oncol 26:2327-35, 2008

27. Toyota M, Ahuja N, Suzuki H, et al: Aberrant methylation in gastric cancer associated with the CpG island methylator phenotype. Cancer Res 59:5438-42, 1999

28. Tanemura A, Terando A M, Sim M S, et al: CpG island methylator phenotype predicts progression of malignant melanoma. Clin Cancer Res 15:1801-7, 2009

29. Peeters K C, Kapiteijn E, van de Velde C J: Managing rectal cancer: the Dutch experience. Colorectal Dis 5:423-6, 2003

30. Kapiteijn E, van de Velde C J: Developments and quality assurance in rectal cancer surgery. Eur J Cancer 38:919-36, 2002

31. Spugnardi M, Tommasi S, Dammann R, et al: Epigenetic inactivation of RAS association domain family protein 1 (RASSF1A) in malignant cutaneous melanoma. Cancer Res 63:1639-43, 2003

32. Herman J G, Graff J R, Myohanen S, et al: Methylation-specific PCR: a novel PCR assay for methylation status of CpG islands. Proc Natl Acad Sci USA 93:9821-6, 1996

33. Umetani N, de Maat M F, Mori T, et al: Synthesis of universal unmethylated control DNA by nested whole genome amplification with phi29 DNA polymerase. Biochem Biophys Res Commun 329:219-23, 2005

34. Liaw A W M: Classification and regression by random forest. R News 2/3:18-22, 2002

35. Little R J A R S: Statistical Analysis with Missing Data. New York, N.Y., Wiley, 2002

All publications cited herein are incorporated by reference in their entirety.

What is claimed is:

1. A method of detecting rectal adenoma or malignancy, comprising:
providing a test biological sample from a subject; and
determining the level of DNA methylation at MINT (methylated-in-tumor) 2, 3, or 31 in the test sample,
wherein the level of methylation at MINT 2, 3, or 31 in the test sample, if higher than that in a normal sample, indicates that the subject is likely to be suffering from rectal adenoma or malignancy.

2. The method of clam 1, wherein the test sample is a rectal tissue sample.

3. A method of predicting the outcome of rectal cancer, comprising:
providing a first sample containing rectal cancer cells from a first subject; and
determining the level of DNA methylation at MINT 3 and the level of DNA methylation at MINT 1, 12, or 17 in the first sample,
wherein the level of DNA methylation at MINT 3 in the first sample, if higher than that in a second sample containing rectal cancer cells from a second subject, and the level of DNA methylation at MINT 1, 12, or 17 in the first sample, if lower than that in the second sample, indicate that the first subject is likely to have an increased risk for distant recurrence, a shorter cancer-specific survival, and a shorter overall survival compared to the second subject.

4. The method of claim 3,
wherein the level of DNA methylation at MINT 3 and the level of DNA methylation at MINT 17 in the first sample are determined, and
wherein the level of DNA methylation at MINT 3 in the first sample, if higher than that in the second sample, and the level of DNA methylation at MINT 17 in the first sample, if lower than that in the second sample, indicate that the first subject is likely to have an increased risk for distant recurrence, a shorter cancer-specific survival, and a shorter overall survival compared to the second subject.

5. The method of claim 3, wherein the first or second subject is node-negative or node-positive.

6. The method of claim 3, wherein the first subject does not receive a radiation therapy prior to a mesorectal excision (ME) for primary rectal cancer.

7. The method of claim 3, wherein the rectal cancer cells are primary rectal cancer cells.

8. The method of claim 3, wherein the first or second subject is suffering from an AJCC Stage I, II, or III rectal cancer.

9. A method of predicting the outcome of rectal cancer, comprising:
providing a first sample containing rectal cancer cells from a first subject; and
determining the level of DNA methylation at MINT 3 and the level of DNA methylation at MINT 1, 12, or 17 in the first sample,
wherein the level of DNA methylation at MINT 3 in the first sample, if lower than that in a second sample containing rectal cancer cells from a second subject, and the level of DNA methylation at MINT 1, 12, or 17 in the first sample, if higher than that in the second sample, indicate that the first subject is likely to have an increased risk for local recurrence compared to the second subject.

10. The method of claim 9,
wherein the level of DNA methylation at MINT 3 and the level of DNA methylation at MINT 17 in the first sample are determined, and
wherein the level of DNA methylation at MINT 3 in the first sample, if lower than that in the second sample, and the level of DNA methylation at MINT 17 in the first sample, if higher than that in the second sample, indicate that the first subject is likely to have an increased risk for local recurrence compared to the second subject.

11. The method of claim 10, wherein the first subject is likely to have an increased risk for local recurrence compared to a third subject that suffers from rectal cancer and receives a radiation therapy prior to an ME for primary rectal cancer.

12. The method of claim 10, wherein the second subject, if node-positive, is likely to have an increased risk for local recurrence compared to a third subject that suffers from rectal cancer but is node-negative.

13. The method of claim 9, wherein the first subject does not receive a radiation therapy prior to an ME for primary rectal cancer.

14. The method of claim 9, wherein the rectal cancer cells are primary rectal cancer cells.

15. The method of claim 9, wherein the first or second subject is suffering from an AJCC Stage I, II, or III rectal cancer.

16. A method of stratifying a rectal cancer treatment, comprising:
providing a biological sample containing primary rectal cancer cells from a subject prior to an ME for primary rectal cancer and a radiation therapy;
determining the level of DNA methylation at MINT 3 and the level of DNA methylation at MINT 17 in the sample; and
stratifying a rectal cancer treatment according to the level of DNA methylation at MINT 3 and the level of DNA methylation at MINT 17 in the sample.

17. The method of claim 16, wherein no radiation therapy is to be given to the subject prior to the ME, if the subject is in a cluster of subjects with rectal cancer that have increased level of DNA methylation at MINT 3 and decreased level of DNA methylation at MINT 17 compared to other clusters of subjects with rectal cancer.

18. The method of claim 17, wherein the clusters are determined using unsupervised random forest (RF) clustering and an expectation-maximization mixture of Gaussians (EM-MoG) algorithm.

19. The method of claim 16, wherein a radiation therapy is to be given to the subject, if the subject is in a cluster of subjects with rectal cancer that have decreased level of DNA methylation at MINT 3 and increased level of DNA methylation at MINT 17 compared to other clusters of subjects with rectal cancer.

20. The method of claim 19, wherein the clusters are determined using unsupervised RF clustering and an EM-MoG algorithm.
